(19) United States
(12) Patent Application Publication
(10) Pub. No.: US 2026/0274839 A1
(43) Pub. Date: Sep. 17, 2026

(54) COMPOUND AND USE THEREOF IN TREATING HUMAN DIHYDROOROTATE DEHYDROGENASE (hDHODH)-MEDIATED DISEASES

(71) Applicant: SICHUAN UNIVERSITY, Chengdu (CN)

(72) Inventors: Yinglan ZHAO, Chengdu (CN); Youfu LUO, Chengdu (CN); Qingxiang SUN, Chengdu (CN)

(73) Assignee: SICHUAN UNIVERSITY, Chengdu (CN)

(21) Appl. No.: 19/472,100

(22) PCT Filed: Apr. 3, 2024
(86) PCT No.: PCT/CN2024/085777

§ 371 Date: Oct. 3, 2025

(30) Foreign Application Priority Data
Apr. 7, 2023 (CN) 202310363167.3

(51) Int. Cl. C07D 471/04; C07B 59/00; C07D 401/04

(52) U.S. Cl. CPC C07D 471/04; C07B 59/002; C07D 401/04; C07B 2200/05

(57) ABSTRACT

A novel small-molecule compound is provided, as shown in Formula I, exhibits pronounced inhibitory activity against an hDHODH enzyme. In the Formula I, Q is a structure being any one selected from the group consisting of:

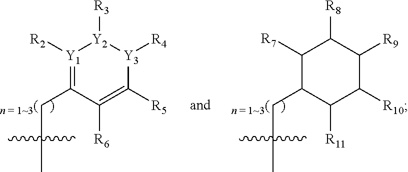

R₂ to R₁₁ are independently selected from the group consisting of hydrogen, halogen, cycloalkyl, alkenyl, alkoxy, a substituted alkyl, and a substituted alkoxy; X₁, X₂, Y₂, and Y₃ are independently selected from the group consisting of C and N, and Y₁ is C; and R₁ is selected from the group consisting of phenyl, a substituted phenyl, pyrimidinyl, and pyridinyl. The compound is suitable for treating various hDHODH-mediated diseases, including but not limited to tumors, autoimmune diseases, and viral infection diseases.

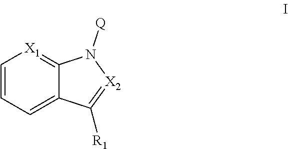

### CROSS REFERENCE TO THE RELATED APPLICATIONS

This application is the national phase entry of International Application No. PCT/CN2024/085777, filed on Apr. 3, 2024, which is based upon and claims priority to Chinese Patent Application No. 202310363167.3, filed on Apr. 7, 2023, the entire contents of which are incorporated herein by reference.

### TECHNICAL FIELD

The present disclosure relates to the technical field of pharmaceutical technology, and particularly discloses a compound and use thereof in the treatment of human dihydroorotate dehydrogenase (hDHODH)-mediated diseases.

### BACKGROUND

Pyrimidine nucleotides are essential building blocks for DNA and RNA synthesis in organisms, and also participate in the biosynthesis of glycoproteins and phospholipids, playing a critical role in cellular proliferation and metabolism. Human dihydroorotate dehydrogenase (hDHODH) catalyzes the fourth step in the de novo pyrimidine synthesis pathway and serves as a key rate-limiting enzyme. Tumor cells exhibit a significantly higher demand for pyrimidine nucleotides compared to normal cells, and predominantly rely on the de novo synthesis pathway.

Inhibition of hDHODH disrupts nascent pyrimidine nucleotide synthesis, thereby impeding the biosynthesis of DNA, RNA, glycoproteins, and phospholipids. This result leads to cell cycle arrest, induces cellular differentiation, and suppresses abnormal cell proliferation, collectively highlighting hDHODH as a promising therapeutic target for antitumor drug development.

Suppressing immune cell proliferation represents an effective therapeutic strategy for treating autoimmune diseases. Inhibiting hDHODH could efficiently block DNA synthesis in activated T and B lymphocytes, curbing their proliferation and reducing inflammatory responses to self-antigens. Furthermore, rapid replication following RNA or DNA viral infections depends on host-cell pyrimidine synthesis. Thus, compounds that targeting the host pyrimidine synthesis pathway could inhibit viral infections. The hDHODH inhibition could effectively attenuate viral replication, positioning it as a potential host-directed antiviral target.

### SUMMARY

The present disclosure provides a small-molecule compound having a structure shown in Formula I:

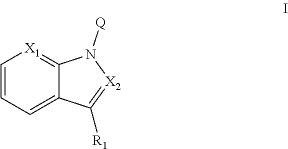

where,
in the Formula I, Q is a structure being any one selected from the group consisting of:

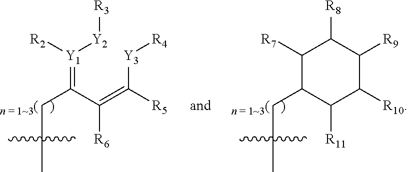

R₂ to R₁₁ are independently selected from the group consisting of hydrogen, halogen, C1-C8 substituted alkyl, cycloalkyl, alkenyl, alkoxy, a substituted alkyl, a substituted alkoxy, trifluoromethyl, and trifluoromethoxy.
X₁, X₂, Y₁, Y₂, and Y₃ are independently selected from the group consisting of C and N.
R₁ is selected from the group consisting of phenyl substituted with 0 to 5 R₁₂, pyrimidinyl, and pyridinyl; R₁₂ is selected from the group consisting of halogen, cyano, C1-C6 alkyl, C1-C6 alkenyl, C3-C9 cycloalkyl, C1-C6 haloalkyl, —CF₃, —NH₂, —NO₂, —SH, —OH, —CH₃COOH, —CH₂OH, a substituted C1-C6 alkoxy, (C3-C9) cycloalkyl (C2-C6)alkynyl, an aldehyde group, and an ester group. The compound has a structure shown in Formula I-1:

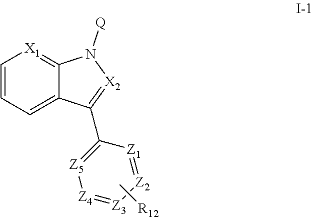

where,
in the Formula I, Q is a structure being any one selected from the group consisting of:

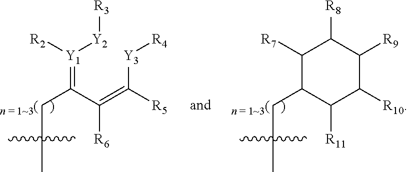

R₂ to R₁₁ are independently selected from the group consisting of hydrogen, halogen, a substituted C1-C8 alkyl, cycloalkyl, alkenyl, alkoxy, the substituted alkyl, the substituted alkoxy, trifluoromethyl, and trifluoromethoxy.
X₁, X₂, Y₁, Y₂, Y₃, Z₁, Z₂, Z₃, Z₄, and Z₅ are independently selected from the group consisting of C and N.
R₁₂ is selected from the group consisting of halogen, cyano, the C1-C6 alkyl, the C1-C6 alkenyl, the C3-C9 cycloalkyl, the C1-C6 haloalkyl, —CF₃, —NH₂, —NO₂, —SH, —OH, —CH₃COOH, —CH₂OH, the substituted C1-C6 alkoxy, (C3-C9) cycloalkyl (C2-C6)alkynyl, an aldehyde group, and an ester group. The compound has a structure shown in Formula I-1.

In some embodiments, Z₁ and Z₅ each are N, and Z₂ to Z₄ each are C; and the compound has a structure shown in Formula I-2:

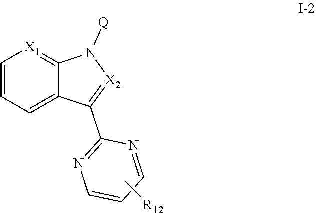

in the Formula I, Q is a structure being any one selected from the group consisting of:

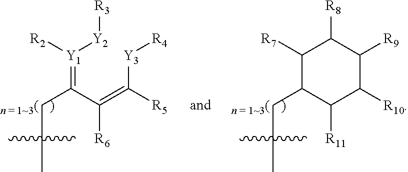

R₂ to R₁₁ are independently selected from the group consisting of hydrogen, halogen, the substituted C1-C8 alkyl, cycloalkyl, alkenyl, alkoxy, substituted alkyl, substituted alkoxy, trifluoromethyl, and trifluoromethoxy.
X₁, X₂, Y₁, Y₂, and Y₃ are independently selected from the group consisting of C and N.
R₁₂ is selected from the group consisting of halogen, cyano, the C1-C6 alkyl, the C1-C6 alkenyl, the C3-C9 cycloalkyl, the C1-C6 haloalkyl, —CF₃, —NH₂, —NO₂, —SH, —OH, —CH₃COOH, —CH₂OH, the substituted alkoxy C1-C6, the (C3-C9) cycloalkyl (C2-C6)alkynyl, an aldehyde group, and an ester group.

In some embodiments, R₁₂ is selected from the group consisting of —F, —Cl, —Br, methyl, ethyl, n-propyl, n-butyl, isopropyl, cyclopropyl, sec-butyl, cyclobutyl, cyclopentyl, isobutyl, 2-methylpentyl, allyl, 2-methylallyl, cyano, —NH₂, —NO₂, —SH, —OH, —CH₃COOH, —CH₂OH, and —CH₃COOCH₃.

In some embodiments, R¹² is any one selected from the group consisting of:

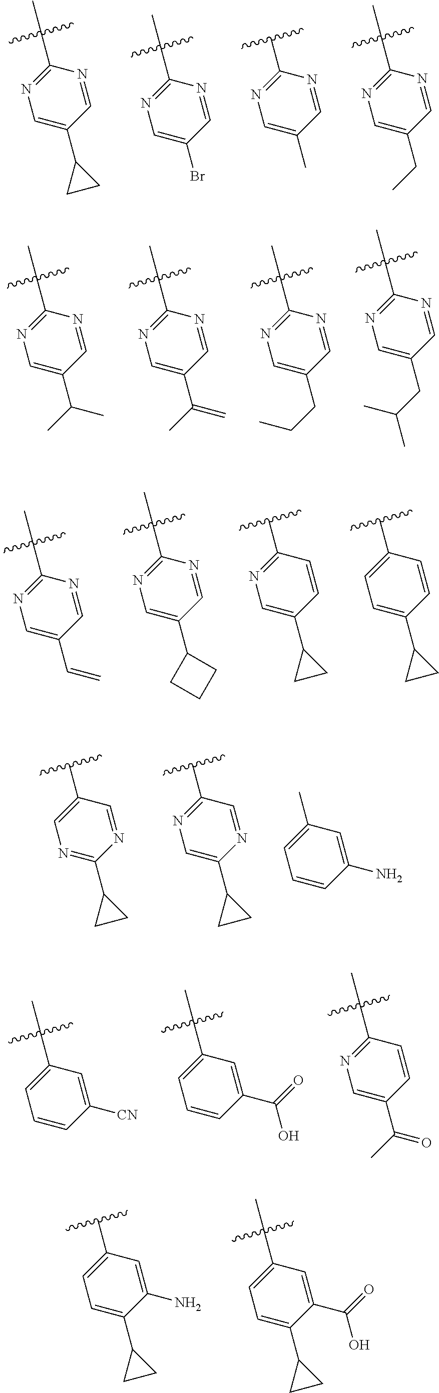

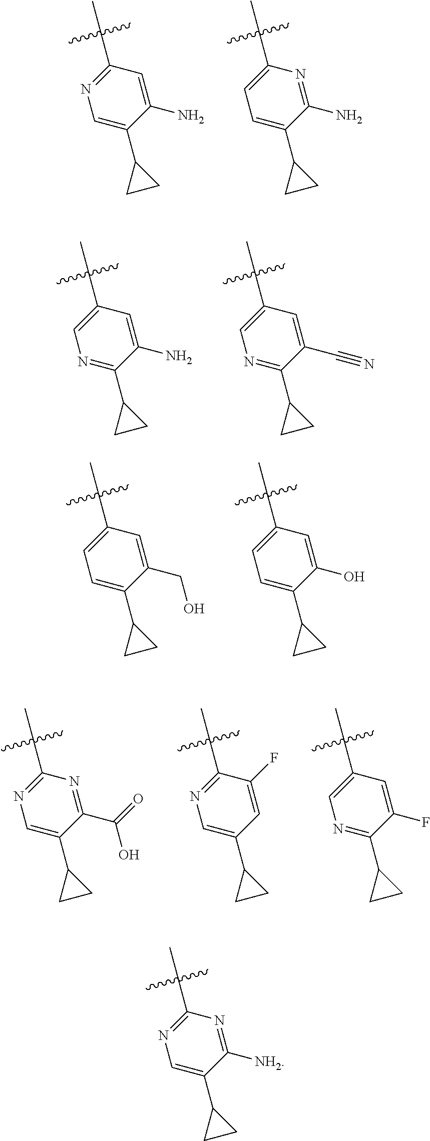

In some embodiments, Q is any one selected from the group consisting of:

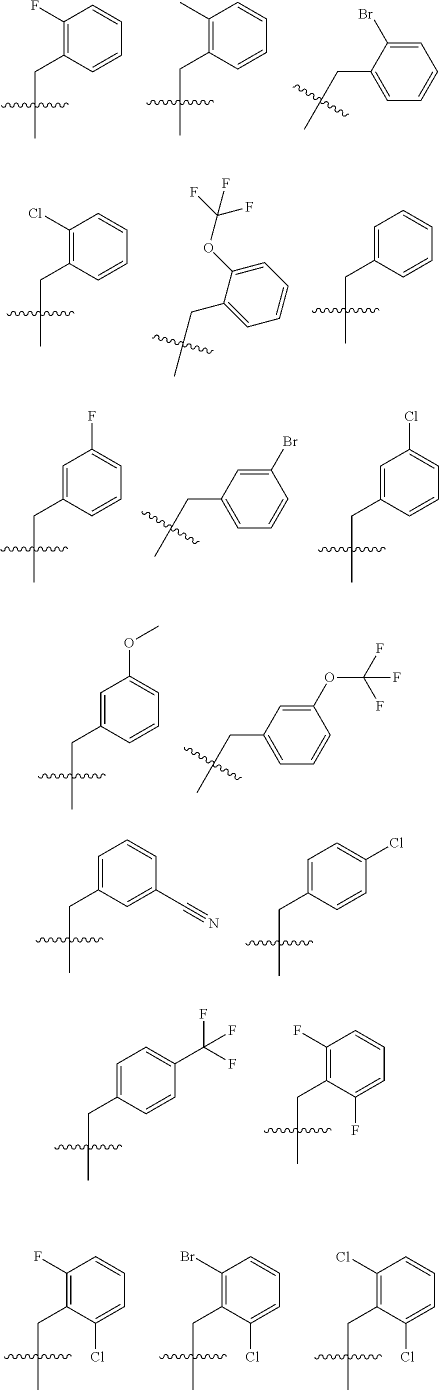

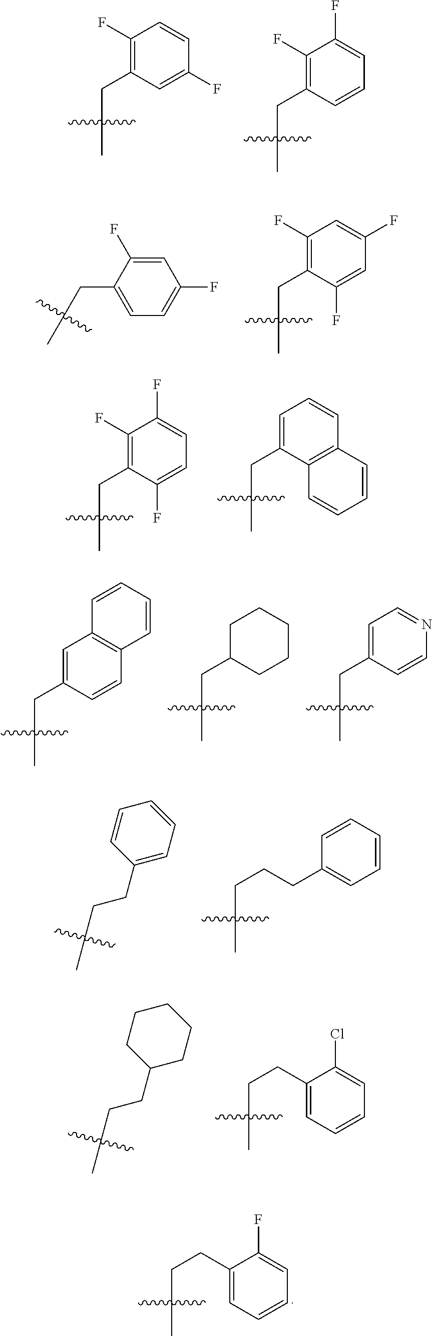

In some embodiments, the compound has a structure being any one selected from the group consisting of:

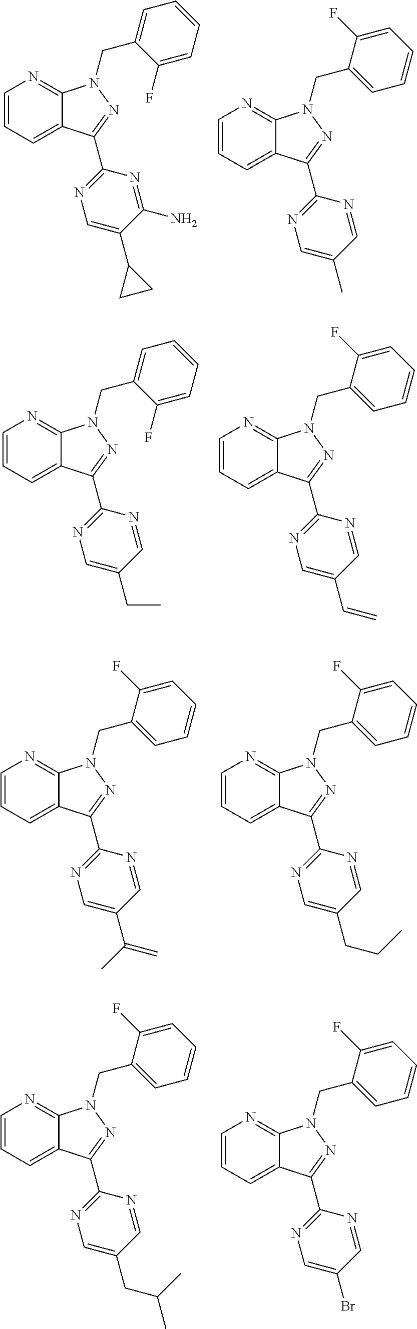

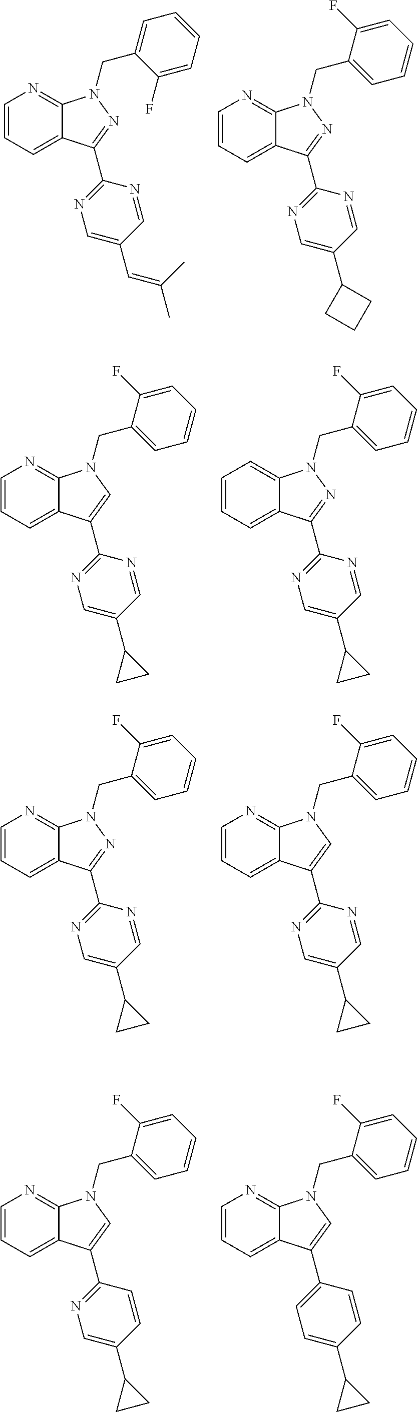

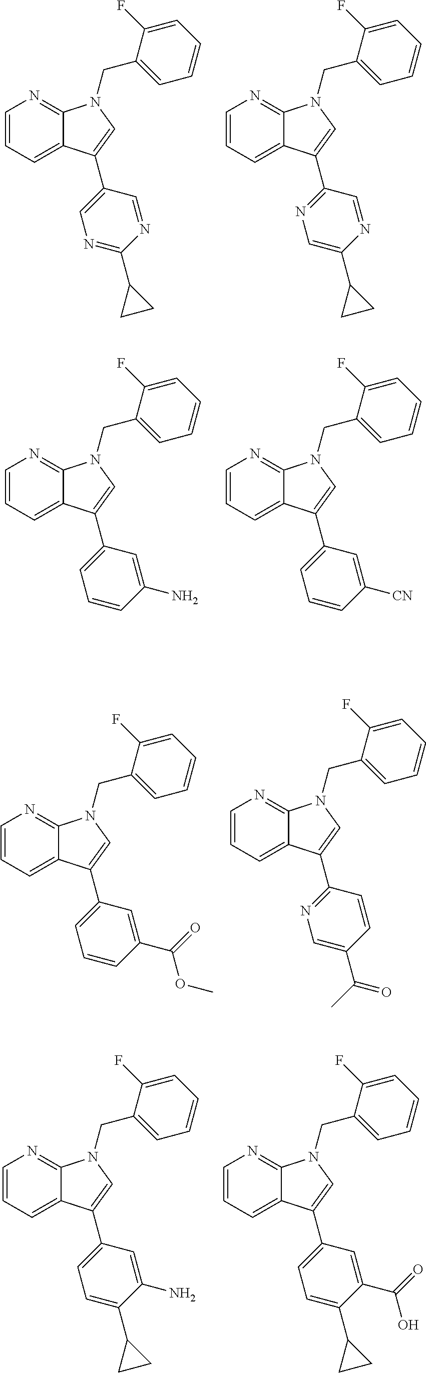

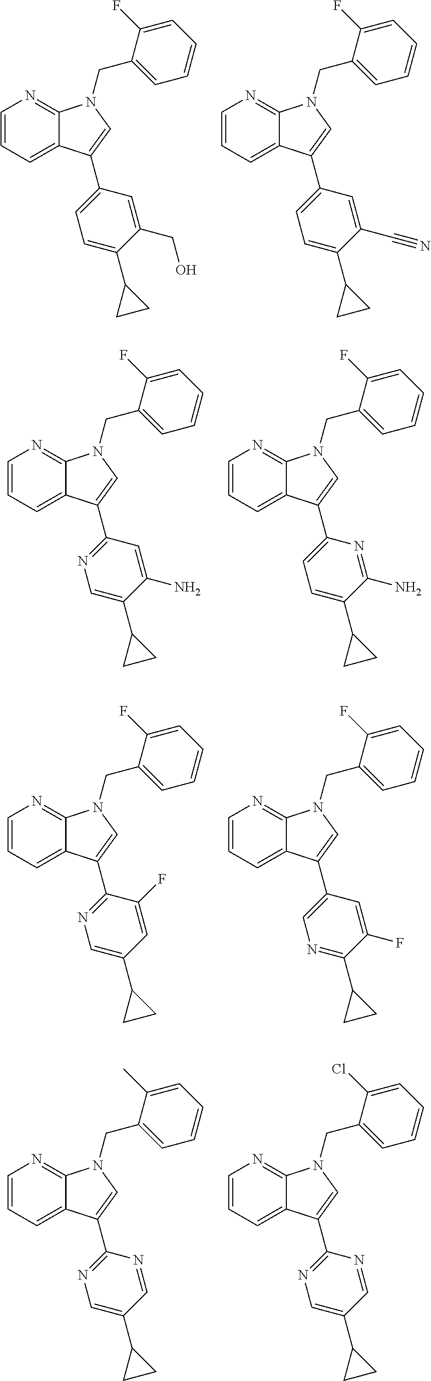

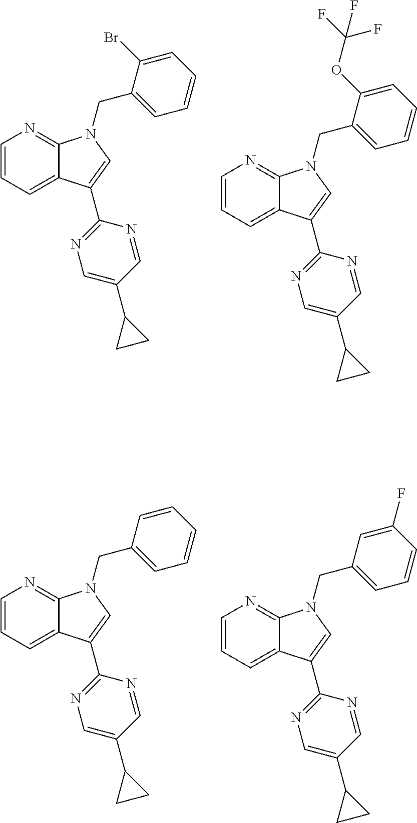 [SMILES: Brc1ccccc1Cn1cc(-c2ncc(C3CC3)cn2)c2cccnc21 ; FC(F)(F)Oc1ccccc1Cn1cc(-c2ncc(C3CC3)cn2)c2cccnc21 ; c1ccc(Cn2cc(-c3ncc(C4CC4)cn3)c3cccnc32)cc1 ; Fc1cccc(Cn2cc(-c3ncc(C4CC4)cn3)c3cccnc32)c1]

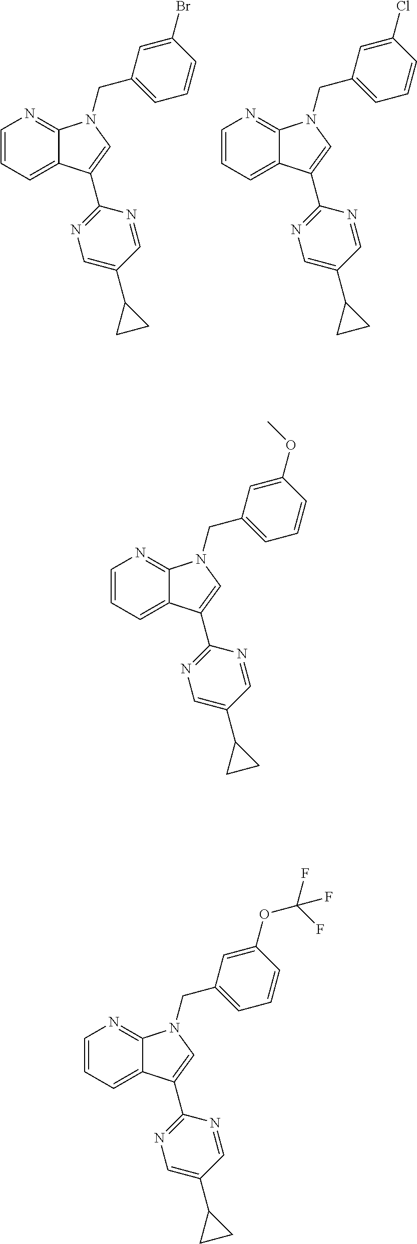 [SMILES: Brc1cccc(Cn2cc(-c3ncc(C4CC4)cn3)c3cccnc32)c1 ; Clc1cccc(Cn2cc(-c3ncc(C4CC4)cn3)c3cccnc32)c1 ; COc1cccc(Cn2cc(-c3ncc(C4CC4)cn3)c3cccnc32)c1 ; FC(F)(F)Oc1cccc(Cn2cc(-c3ncc(C4CC4)cn3)c3cccnc32)c1]

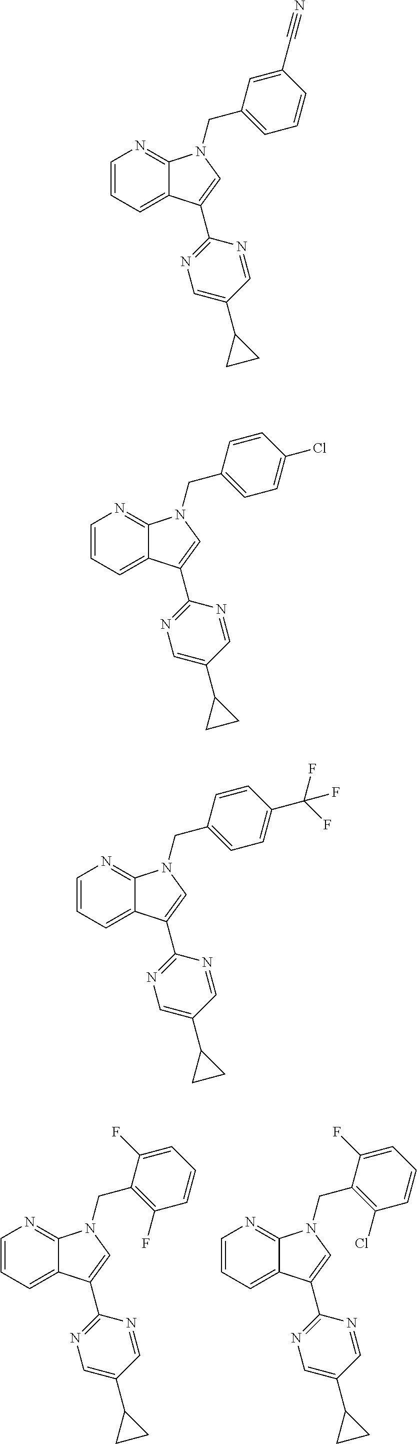 [SMILES: N#Cc1cccc(Cn2cc(-c3ncc(C4CC4)cn3)c3cccnc32)c1 ; Clc1ccc(Cn2cc(-c3ncc(C4CC4)cn3)c3cccnc32)cc1 ; FC(F)(F)c1ccc(Cn2cc(-c3ncc(C4CC4)cn3)c3cccnc32)cc1 ; Fc1cccc(F)c1Cn1cc(-c2ncc(C3CC3)cn2)c2cccnc21 ; Fc1cccc(Cl)c1Cn1cc(-c2ncc(C3CC3)cn2)c2cccnc21]

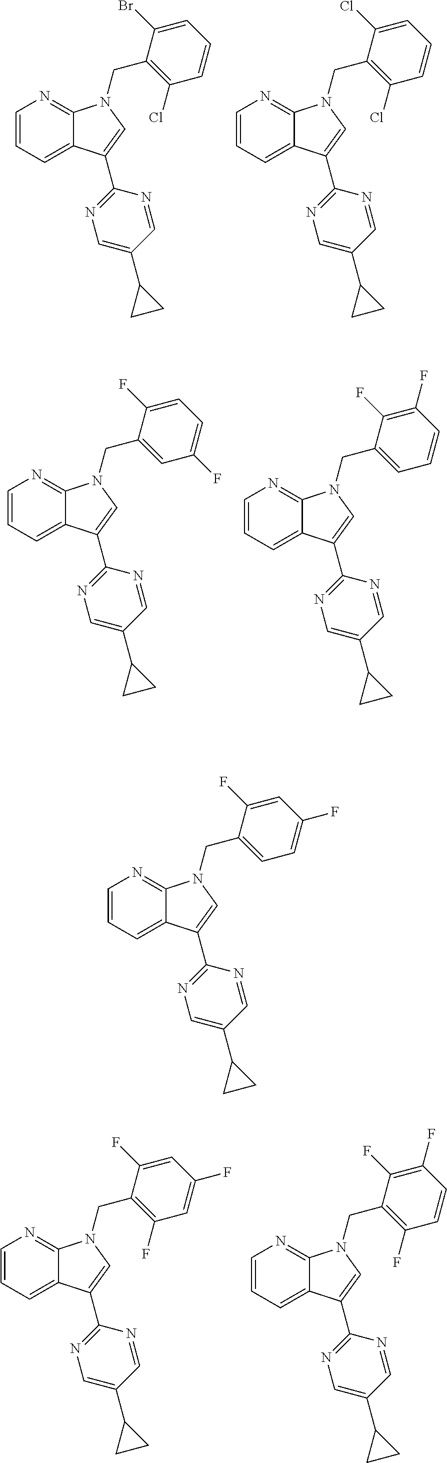

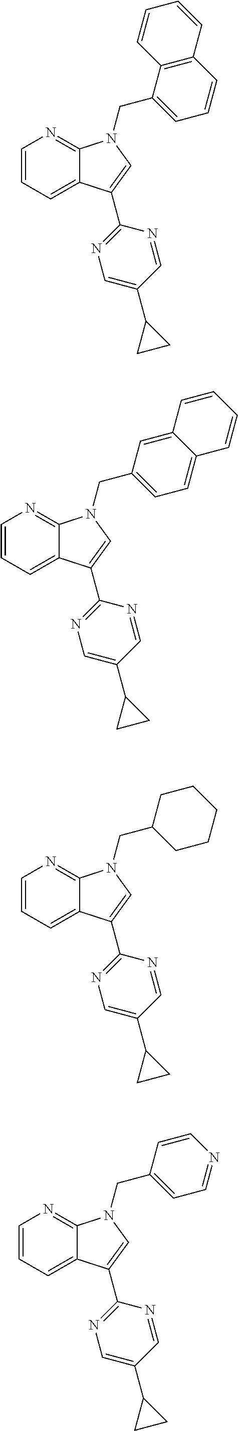 [SMILES: c1ccc2c(Cn3cc(-c4ncc(C5CC5)cn4)c4cccnc43)cccc2c1 ; c1ccc2cc(Cn3cc(-c4ncc(C5CC5)cn4)c4cccnc43)ccc2c1 ; c1cnc2c(c1)c(-c1ncc(C3CC3)cn1)cn2CC1CCCCC1 ; c1cnc2c(c1)c(-c1ncc(C3CC3)cn1)cn2Cc1ccncc1]

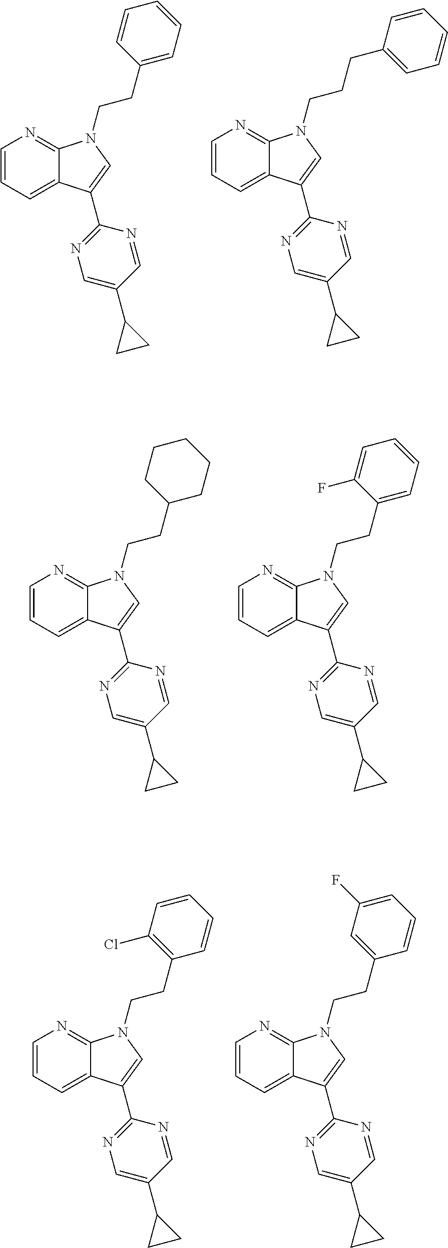 [SMILES: c1ccc(CCn2cc(-c3ncc(C4CC4)cn3)c3cccnc32)cc1 ; c1ccc(CCCn2cc(-c3ncc(C4CC4)cn3)c3cccnc32)cc1 ; c1cnc2c(c1)c(-c1ncc(C3CC3)cn1)cn2CCC1CCCCC1 ; Fc1ccccc1CCn1cc(-c2ncc(C3CC3)cn2)c2cccnc21 ; Clc1ccccc1CCn1cc(-c2ncc(C3CC3)cn2)c2cccnc21 ; Fc1cccc(CCn2cc(-c3ncc(C4CC4)cn3)c3cccnc32)c1]

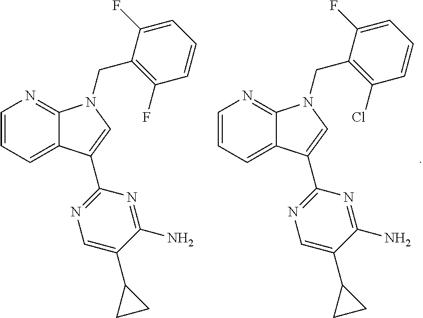 [SMILES: Nc1nc(-c2cn(Cc3c(F)cccc3F)c3ncccc23)ncc1C1CC1 ; Nc1nc(-c2cn(Cc3c(F)cccc3Cl)c3ncccc23)ncc1C1CC1]

The present disclosure further provides a method for preparing the compound.

The method, as shown in Synthetic Route 1, includes the following steps:

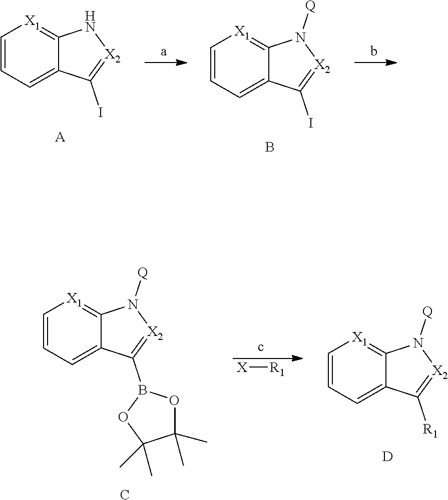

(a) subjecting a starting material A and a halogen-substituted benzyl to reaction under an alkaline environment to form an intermediate B;
(b) subjecting the intermediate B and bis(pinacolato)diboron to Suzuki coupling to obtain an intermediate C; and
(c) subjecting the intermediate C and a halogen-containing a substituted phenyl, a substituted pyridinyl, or a substituted pyrimidinyl to Suzuki coupling to obtain a final product D.

The present disclosure further provides an isotopic substitute of any atom or multiple atoms of the compound as described above, where an isotope in the isotopic substitute is deuterium.

The present disclosure further provides a pharmaceutically acceptable hydrate, a solvate or a polymorph, and a salt of the novel small-molecule compound as described above.

The present disclosure further provides use of the novel small-molecule compound as described above in manufacture of a drug for treating an hDHODH-mediated disease.

In the present disclosure, the hDHODH-mediated disease is selected from the group consisting of a tumor, an autoimmune disease, and a viral infection.

### DETAILED DESCRIPTION OF THE EMBODIMENTS

The features and properties of the present application are further described in detail below in conjunction with examples. The examples are for illustration only and do not limit the present disclosure in any way.

### Example 1: Preparation of Representative Intermediate B1

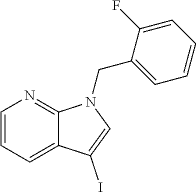 [SMILES: Fc1ccccc1Cn1cc(I)c2cccnc21]

3-Iodo-7-azaindole (1 equivalent (equiv)) as a starting material was placed in a two-necked flask. DMF was added thereto as a solvent. A resulting mixture was cooled to 0° C. under stirring to obtain a cooled mixture. Sodium hydride powder (1.1 equiv) was added slowly to the cooled mixture, and the reaction was continued at 0° C. for 1 h. Subsequently, 2-fluorobenzyl bromide (1.05 equiv) was added thereto. After completion of the addition, an obtained mixture was stirred at room temperature for 4 h. After confirmation of reaction completion by TLC monitoring, a reaction product was extracted three times with ethyl acetate and water, and organic phases were combined. A combined organic phase was washed with water and saturated brine in sequence, and then dried with anhydrous sodium sulfate. The sodium sulfate was removed by filtration. A resulting filtrate was concentrated under reduced pressure, and then purified by flash chromatography on a Biotage Isolera Prime system equipped with 200 to 300 mesh silica gel column, to obtain the target compound as a pale-yellow solid with a yield of 93%, 1H NMR (400 MHz, DMSO) δ 8.40-8.25 (m, 1H), 7.82 (s, 1H), 7.75-7.64 (m, 1H), 7.45-7.27 (m, 1H), 7.27-7.15 (m, 2H), 7.10 (dd, J=11.8, 6.4 Hz, 2H), 5.56 (s, 2H). ¹³C NMR (100 MHz, DMSO) δ 160.31 (d, J=245.5 Hz), 147.31, 144.42, 133.54, 130.24 (d, J=8.1 Hz), 130.11 (d, J=4.0 Hz), 129.22, 125.18, 125.05 (d, J=4.0 Hz), 122.92, 117.37, 115.88 (d, J=20.9 Hz), 54.94.

### Example 2: Preparation of Representative Intermediate C1

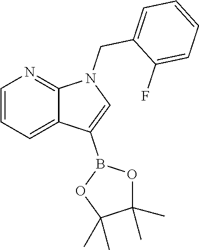 [SMILES: CC1(C)OB(c2cn(Cc3ccccc3F)c3ncccc23)OC1(C)C]

Compound B1 (1 equiv) and bis(pinacolato)diboron (1.1 equiv) were added to a two-necked flask. A mixture of dioxane and water (3:1) was added thereto as a solvent. A reaction system was purged 2 times with N₂, and then Pd(dppf) Cl₂·CH₂Cl₂ (0.1 equiv) and cesium carbonate (3 equiv) was added to the reaction system under N₂ atmosphere. After completion of the addition, a resulting system was purged 3 more times with N₂ and then heated at 90° C. for 8 h under N₂ protection. After confirmation of reaction completion by TLC monitoring, an obtained reaction system was cooled to room temperature and extracted three times with dichloromethane and water, and organic phases were combined. A combined organic phase was washed with water and saturated brine in sequence, and dried with anhydrous sodium sulfate. The desiccant was removed by filtration. A resulting filtrate was concentrated under reduced pressure, and then purified by flash chromatography on a Biotage Isolera Prime system equipped with 200 to 300 mesh silica gel column, to obtain the target compound as a white solid with a yield of 76%, 1H NMR (400 MHz, DMSO) & 8.30 (d, J=4.2 Hz, 1H), 8.14 (d, J=7.6 Hz, 1H), 7.91 (s, 1H), 7.41-7.03 (m, 5H), 5.58 (s, 2H), 1.30 (s, 12H). 13C NMR (100 MHz, DMSO) & 160.40 (d, J=245.4 Hz), 148.77, 143.47, 138.84, 130.32, 130.29, 130.21, 125.13 (d, J=5.8 Hz), 125.04 (d, J=5.6 Hz), 124.17, 117.24, 115.98, 115.77, 83.27 (2C), 41.91 (d, J=4.2 Hz), 25.18 (4C).

### Example 3: Preparation of Representative Final Product D1

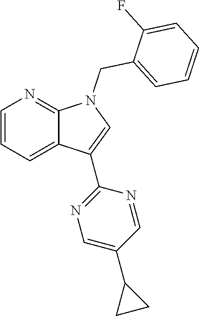 [SMILES: Fc1ccccc1Cn1cc(-c2ncc(C3CC3)cn2)c2cccnc21]

Compound C1 (1 equiv) and 5-cyclopropyl-2-chloropyrimidine (1.1 equiv) were added to a two-necked flask. A mixture of dioxane and water (3:1) was added as a solvent thereto. A reaction system was purged 2 times with N₂, and then Pd(dppf)Cl₂·CH₂Cl₂ (0.1 equiv) and cesium carbonate (3 equiv) was added to the reaction system under N₂ atmosphere. After completion of the addition, a resulting system was purged 3 more times with N₂ and then heated at 90° C. for 8 h under N₂ protection. After confirmation of reaction completion by TLC monitoring, an obtained reaction system was cooled to room temperature and extracted three times with dichloromethane and water, and organic phases were combined. A combined organic phase was washed with water and saturated brine in sequence, and dried with anhydrous sodium sulfate. The desiccant was removed by filtration. A resulting filtrate was concentrated under reduced pressure, and then purified by flash chromatography on a Biotage Isolera Prime system equipped with 200 to 300 mesh silica gel column, to obtain the target compound as a white solid with a yield of 82%, 1H NMR (400 MHz, DMSO) δ 8.79 (d, J=7.9 Hz, 1H), 8.56 (d, J=6.2 Hz, 2H), 8.44-8.27 (m, 2H), 7.34 (s, 1H), 7.31-7.21 (m, 2H), 7.21-7.09 (m, 2H), 5.65 (s, 2H), 1.94 (s, 1H), 1.03 (dd, J=5.4, 2.9 Hz, 2H), 0.94-0.69 (m, 2H). ¹³C NMR (100 MHz, DMSO) δ 160.46 (d, J=245.2 Hz), 159.29 (d, J=244.0 Hz), 155.04 (2C), 148.25, 143.95, 143.95, 133.12, 131.61, 130.37 (d, J=3.3 Hz), 130.31, 125.14 (d, J=3.5 Hz), 124.92, 118.74, 117.80, 115.94 (d, J=20.9 Hz), 113.60, 42.07, 11.02, 9.24 (2C). HRMS m/z (ESI) calculated for C₂₁H₁₇FN₄, 345.1515, [M+H]⁺, found: 345.1505.

All remaining final products were prepared according to the methods described above.

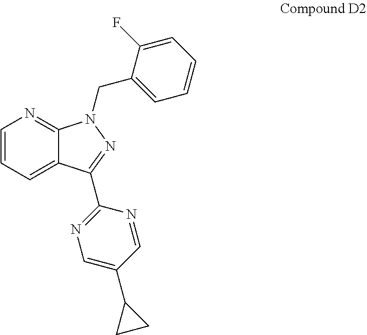 [SMILES: Fc1ccccc1Cn1nc(-c2ncc(C3CC3)cn2)c2cccnc21]

¹H NMR (400 MHz, DMSO) δ 8.85 (dd, J=8.1, 1.6 Hz, 1H), 8.71 (s, 2H), 8.67 (dd, J=4.5, 1.6 Hz, 1H), 7.41 (dd, J=8.1, 4.5 Hz, 1H), 7.39-7.30 (m, 1H), 7.29-7.19 (m, 2H), 7.15 (td, J=7.6, 1.0 Hz, 1H), 5.86 (s, 2H), 2.09-1.94 (m, 1H), 1.09 (qd, J=6.6, 3.4 Hz, 2H), 0.99-0.82 (m, 2H). ¹³C NMR (100 MHz, DMSO) § 160.44 (d, J=246.1 Hz), 158.02, 155.28 (2C), 151.38, 149.87, 141.28, 136.05, 133.02, 130.74 (d, J=3.8 Hz), 130.46 (d, J=8.2 Hz), 125.10 (d, J=3.1 Hz), 124.30 (d, J=14.7 Hz), 119.06, 115.97 (d, J=21.3 Hz), 114.77, 44.55, 11.17, 9.84 (2C). HRMS m/z (ESI) calculated for 346.1468, [M+H]⁺, found: 346.1462.

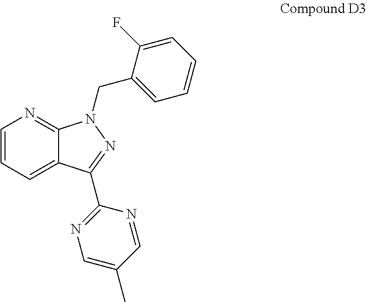 [SMILES: Cc1cnc(-c2nn(Cc3ccccc3F)c3ncccc23)nc1]

1H NMR (400 MHz, DMSO) δ 8.87 (dd, J=8.1, 1.6 Hz, 1H), 8.81 (d, J=0.4 Hz, 2H), 8.68 (dd, J=4.5, 1.6 Hz, 1H), 7.42 (dd, J=8.1, 4.5 Hz, 1H), 7.36 (s, 1H), 7.24 (t, J=6.7 Hz, 2H), 7.19-7.12 (m, 1H), 5.86 (s, 2H), 2.35 (s, 3H). ¹³C NMR (100 MHz, DMSO) δ 159.98 (d, J=341.6 Hz), 158.07 (2C), 151.38, 149.92, 141.28, 133.09, 130.77 (d, J=3.7 Hz), 130.50 (d, J=8.1 Hz), 130.03, 125.14 (d, J=3.3 Hz), 124.30 (d, J=14.7 Hz), 119.12, 116.10, 115.89, 114.78. HRMS m/z (ESI) calculated for C₁₈H₁₄FN₅, 320.1311, [M+H]⁺, found: 320.1300.

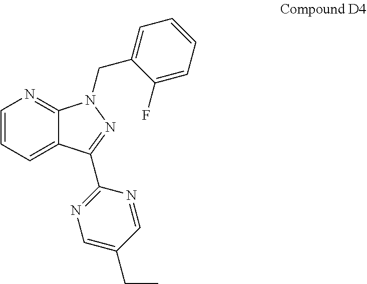 [SMILES: CCc1cnc(-c2nn(Cc3ccccc3F)c3ncccc23)nc1]

¹H NMR (400 MHz, DMSO) δ 8.99-8.77 (m, 3H), 8.72-8.64 (m, 1H), 7.49-7.33 (m, 2H), 7.24 (t, J=8.5 Hz, 2H), 7.16 (t, J=7.4 Hz, 1H), 5.87 (s, 2H), 2.70 (q, J=7.3 Hz, 2H), 1.26 (t, J=7.5 Hz, 3H). ¹³C NMR (100 MHz, DMSO) δ 160.43 (d, J=245.6 Hz), 158.54 (2C), 157.39, 151.37, 149.90, 135.64, 133.06, 130.72 (d, J=3.7 Hz), 130.47 (d, J=8.1 Hz), 125.11 (d, J=3.5 Hz), 124.30 (d, J=14.8 Hz), 119.12, 116.08, 115.87, 114.80, 44.58 (d, J=4.4 Hz), 23.20, 15.30. HRMS m/z (ESI) calculated for C₁₉H₁₆FN₅, 334.1468, [M+H]⁺, found: 334.1465.

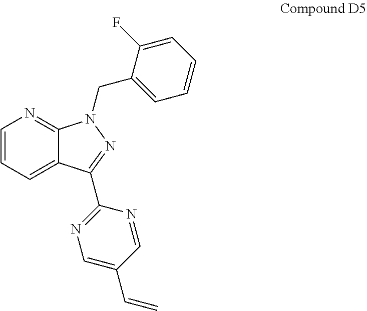 [SMILES: C=Cc1cnc(-c2nn(Cc3ccccc3F)c3ncccc23)nc1]

¹H NMR (400 MHz, DMSO) δ 9.08 (s, 2H), 8.94-8.84 (m, 1H), 8.69 (dd, J=4.5, 1.6 Hz, 1H), 7.44 (dd, J=8.1, 4.5 Hz, 1H), 7.37 (ddd, J=15.4, 5.5, 1.8 Hz, 1H), 7.30-7.21 (m, 2H), 7.16 (td, J=7.6, 1.0 Hz, 1H), 6.83 (dd, J=17.8, 11.2 Hz, 1H), 6.20 (d, J=17.8 Hz, 1H), 5.88 (s, 2H), 5.56 (d, J=11.3 Hz, 1H). ¹³C NMR (100 MHz, DMSO) δ 160.46 (d, J=246.0 Hz), 159.39 (2C), 155.43, 151.40, 149.99, 141.02, 133.06, 130.79 (d, J=3.4 Hz), 130.52 (d, J=8.2 Hz), 129.31, 125.13 (d, J=3.5 Hz), 124.23 (d, J=14.7 Hz), 119.20 (d, J=18.4 Hz), 116.00 (d, J=21.0 Hz), 114.94, 73.98, 44.68 (d, J=4.2 Hz), 25.43. HRMS m/z (ESI) calculated for C₁₉H₁₄FN₅, 332.1311, [M+H]⁺, found: 332.1303.

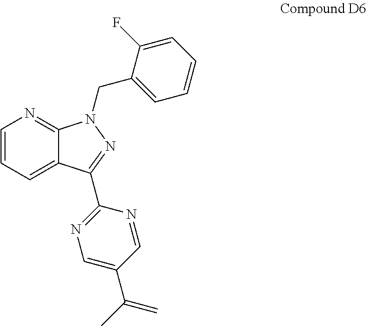 [SMILES: C=C(C)c1cnc(-c2nn(Cc3ccccc3F)c3ncccc23)nc1]

¹H NMR (400 MHz, DMSO) δ 9.08 (d, J=13.4 Hz, 2H), 8.93-8.83 (m, 1H), 8.69 (dd, J=4.5, 1.6 Hz, 1H), 7.44 (dd, J=8.1, 4.5 Hz, 1H), 7.37 (ddd, J=15.4, 5.5, 1.8 Hz, 1H), 7.30-7.21 (m, 2H), 7.16 (td, J=7.5, 1.1 Hz, 1H), 5.88 (s, 2H), 5.76 (d, J=4.6 Hz, 1H), 5.33 (s, 1H), 2.19 (d, J=8.8 Hz, 3H). ¹³C NMR (100 MHz, DMSO) δ 160.50 (d, J=237.6 Hz), 154.67 (2C), 151.39, 149.98, 141.00, 137.77, 137.77, 133.00, 131.86, 130.79 (d, J=3.9 Hz), 130.52 (d, J=8.2 Hz), 125.12 (d, J=3.5 Hz), 124.23 (d, J=14.7 Hz), 119.27, 116.19 (d, J=19.0 Hz), 115.89, 114.94, 44.67 (d, J=3.8 Hz), 21.02. HRMS m/z (ESI) calculated for C₂₀H₁₆FN₅, 346.1468, [M+H]⁺, found: 346.1457.

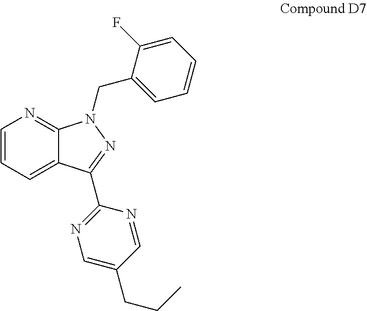 [SMILES: CCCc1cnc(-c2nn(Cc3ccccc3F)c3ncccc23)nc1]

¹H NMR (400 MHz, DMSO) δ 8.91-8.86 (m, 1H), 8.82 (s, 2H), 8.68 (dd, J=4.5, 1.5 Hz, 1H), 7.42 (dd, J=8.1, 4.5 Hz, 1H), 7.37 (d, J=7.0 Hz, 1H), 7.28-7.21 (m, 2H), 7.16 (d, J=7.5 Hz, 1H), 5.87 (s, 2H), 2.63 (t, J=7.6 Hz, 2H), 1.66 (dd, J=15.0, 7.4 Hz, 2H), 0.93 (t, J=7.3 Hz, 3H). ¹³C NMR (10 MHz, DMSO) δ 160.44 (d, J=246.1 Hz), 158.59 (2C), 157.74, 151.37, 149.87, 141.27, 134.06, 133.05, 130.73 (d, J=3.8 Hz), 130.46 (d, J=8.1 Hz), 125.09 (d, J=3.5 Hz), 124.30 (d, J=14.7 Hz), 119.08, 115.96 (d, J=21.0 Hz), 114.80, 44.57 (d, J=4.1 Hz), 31.80, 23.86, 13.85. HRMS m/z (ESI) calculated for C₂₀H₁₈FN₅, 348.1624, [M+H]⁺, found: 348.1612.

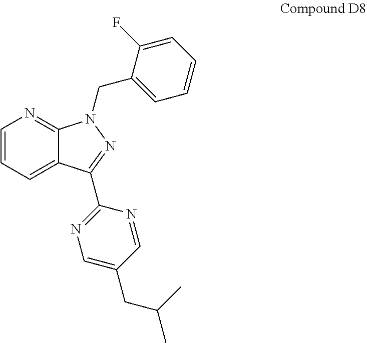 [SMILES: CC(C)Cc1cnc(-c2nn(Cc3ccccc3F)c3ncccc23)nc1]

¹H NMR (400 MHz, DMSO) δ 8.89 (dd, J=8.1, 1.6 Hz, 1H), 8.79 (s, 2H), 8.68 (dd, J=4.5, 1.6 Hz, 1H), 7.43 (dd, J=8.1, 4.5 Hz, 1H), 7.39-7.33 (m, 1H), 7.29-7.20 (m, 2H), 7.19-7.10 (m, 1H), 5.87 (s, 2H), 2.54 (d, J=7.1 Hz, 2H), 1.97-1.87 (m, 1H), 0.91 (d, J=6.6 Hz, 6H). 13C NMR (100 MHz, DMSO) δ 160.46 (d, J=246.2 Hz), 158.64, 158.18 (2C), 151.40, 149.91, 141.28, 133.08, 133.05, 130.78, 130.48 (d, J=8.0 Hz), 125.13, 124.29 (d, J=14.7 Hz), 119.12, 115.98 (d, J=20.2 Hz), 114.82, 44.60 (d, J=4.0 Hz), 38.82, 29.58, 22.30 (2C). HRMS m/z (ESI) calculated for C₂₁H₂₀FN₅, 362.1781, [M+H]⁺, found: 362.1768.

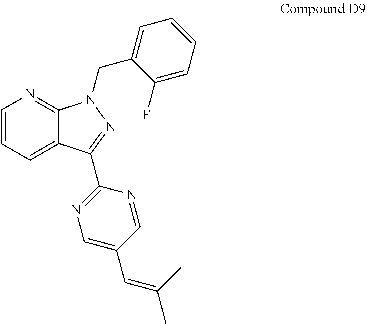 [SMILES: CC(C)=Cc1cnc(-c2nn(Cc3ccccc3F)c3ncccc23)nc1]

¹H NMR (400 MHz, DMSO) δ 8.89 (d, J=8.1 Hz, 1H), 8.83 (s, 2H), 8.68 (d, J=4.3 Hz, 1H), 7.49-7.32 (m, 2H), 7.32-7.21 (m, 2H), 7.16 (t, J=7.4 Hz, 1H), 6.28 (s, 1H), 5.87 (s, 2H), 1.95 (d, J=12.9 Hz, 6H). ¹³C NMR (100 MHz, DMSO) & 160.46 (d, J=246.1 Hz), 157.66, 156.93 (2C), 151.38, 149.93, 141.08 (d, J=9.8 Hz), 133.07, 130.81 (d, J=3.9 Hz), 130.51 (d, J=8.0 Hz), 130.40, 125.12 (d, J=3.5 Hz), 124.26 (d, J=14.6 Hz), 119.17, 118.64, 115.98 (d, J=21.0 Hz), 114.86, 100.00, 44.60, 27.36, 20.00 (2C). HRMS m/z (ESI) calculated for C₂₁H₁₈FN₅, 360.1624, [M+H]⁺, found: 360.1618.

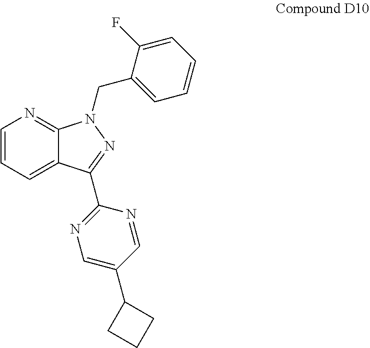 [SMILES: Fc1ccccc1Cn1nc(-c2ncc(C3CCC3)cn2)c2cccnc21]

¹H NMR (400 MHz, CDCl₃) δ 8.96 (dd, J=8.1, 1.3 Hz, 1H), 8.84 (s, 1H), 8.75 (s, 1H), 8.61 (dd, J=4.5, 1.4 Hz, 1H), 7.32-7.27 (m, 1H), 7.26-7.17 (m, 1H), 7.14-6.86 (m, 3H), 5.97 (s, 2H), 3.61 (s, 1H), 2.55-1.85 (m, 5H). HRMS m/z (ESI) calculated for C₂₁H₁₈FN₅, 382.1444, [M+Na]⁺, found: 382.1438.

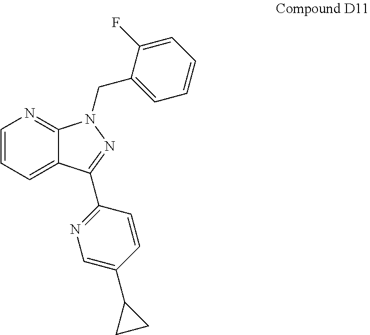 [SMILES: Fc1ccccc1Cn1nc(-c2ccc(C3CC3)cn2)c2cccnc21]

¹H NMR (400 MHz, DMSO) δ 8.76 (d, J=7.9 Hz, 1H), 8.44 (s, 1H), 8.33 (d, J=4.5 Hz, 1H), 8.28 (s, 1H), 7.73 (d, J=8.2 Hz, 1H), 7.41 (d, J=8.2 Hz, 1H), 7.38-7.29 (m, 1H), 7.24 (dd, J=10.9, 7.2 Hz, 2H), 7.16-7.06 (m, 2H), 5.61 (s, 2H), 1.96 (s, 1H), 1.08-0.93 (m, 2H), 0.75 (d, J=5.0 Hz, 2H). ¹³C NMR (100 MHz, DMSO) δ 160.35 (d, J=245.2 Hz), 151.73, 148.20, 147.74, 143.74, 136.23, 133.25, 130.73, 130.20 (d, J=8.0 Hz), 130.08 (d, J=4.0 Hz), 128.61, 125.23 (d, J=14.9 Hz), 125.11 (d, J=3.4 Hz), 119.54, 118.42, 117.32, 115.89 (d, J=20.9 Hz), 114.37, 41.80, 13.01, 9.51 (2C). HRMS m/z (ESI) calculated for C₂₂H₁₈FN₃, 344.1563, [M+H]⁺, found: 344.1555.

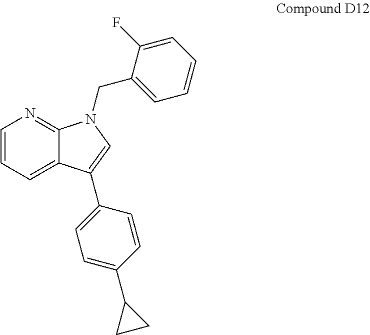 [SMILES: Fc1ccccc1Cn1cc(-c2ccc(C3CC3)cc2)c2cccnc21]

¹H NMR (400 MHz, DMSO) § 8.30 (ddd, J=9.5, 6.3, 1.5 Hz, 2H), 7.92 (s, 1H), 7.66-7.51 (m, 2H), 7.39-7.29 (m, 1H), 7.27-7.04 (m, 6H), 5.59 (s, 2H), 1.99-1.90 (m, 1H), 1.04-0.91 (m, 2H), 0.79-0.64 (m, 2H). ¹³C NMR (100 MHz, DMSO) δ 160.33 (d, J=245.2 Hz), 148.03, 143.53, 141.90, 131.89, 130.13 (d, J=8.1 Hz), 130.05 (d, J=4.0 Hz), 128.55, 126.68, 126.56, 126.42, 125.38 (d, J=14.8 Hz), 125.11, 118.14, 116.94, 115.87 (d, J=21.0 Hz), 114.60, 41.66, 15.31, 9.76 (2C). HRMS m/z (ESI) calculated for C₂₃H₁₉FN₂, 343.161, [M+H]⁺, found:

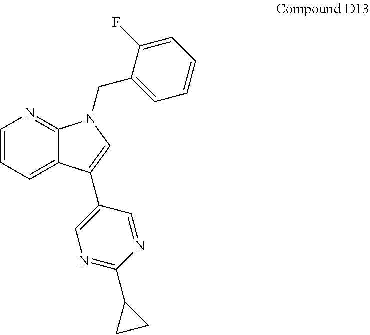 [SMILES: Fc1ccccc1Cn1cc(-c2cnc(C3CC3)nc2)c2cccnc21]

¹H NMR (400 MHz, DMSO) δ 9.00 (s, 2H), 8.48-8.30 (m, 2H), 8.18 (s, 1H), 7.40-7.29 (m, 1H), 7.23 (dd, J=9.7, 4.7 Hz, 2H), 7.13 (d, J=4.2 Hz, 2H), 5.61 (s, 2H), 2.24 (dd, J=7.4, 4.9 Hz, 1H), 1.16-0.92 (m, 4H). ¹³C NMR (100 MHz, DMSO) § 168.68, 160.34 (d, J=245.4 Hz), 154.29 (2C), 147.99, 144.01, 130.22 (d, J=8.0 Hz), 130.10 (d, J=4.0 Hz), 128.77, 127.75, 125.73, 125.14 (d, J=3.5 Hz), 125.05 (d, J=7.8 Hz), 117.78, 117.37, 115.89 (d, J=20.9 Hz), 108.18, 41.86 (d, J=4.5 Hz), 18.15, 10.65 (2C). HRMS m/z (ESI) calculated for C₂₁H₁₇FN₄, 345.1515, [M+H]⁺, found: 345.1504.

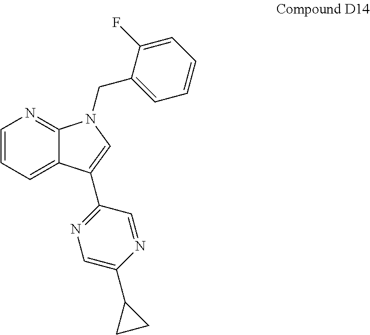 [SMILES: Fc1ccccc1Cn1cc(-c2cnc(C3CC3)cn2)c2cccnc21]

¹H NMR (400 MHz, DMSO) δ 8.97 (d, J=1.3 Hz, 1H), 8.71 (dd, J=7.9, 1.5 Hz, 1H), 8.60 (d, J=1.3 Hz, 1H), 8.43 (s, 1H), 8.36 (dd, J=4.6, 1.4 Hz, 1H), 7.34 (dd, J=5.7, 2.9 Hz, 1H), 7.29-7.20 (m, 2H), 7.13 (dd, J=5.1, 3.0 Hz, 2H), 5.63 (s, 2H), 2.24-2.15 (m, 1H), 1.11-1.01 (m, 2H), 1.00-0.88 (m, 2H). ¹³C NMR (100 MHz, DMSO) δ 160.35 (d, J=245.3 Hz), 154.13, 148.17, 147.24, 144.12, 142.46, 140.81, 130.56, 130.26 (d, J=8.2 Hz), 130.07 (d, J=3.9 Hz), 129.19, 125.14 (d, J=3.4 Hz), 124.98, 118.24, 117.65, 115.92 (d, J=20.9 Hz), 111.49, 41.97 (d, J=3.9 Hz), 14.50, 10.13 (2C). HRMS m/z (ESI) calculated for C₂₁H₁₇FN₄, 345.1515, [M+H]⁺, found: 345.1504.

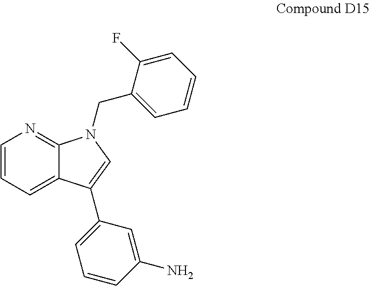 [SMILES: Nc1cccc(-c2cn(Cc3ccccc3F)c3ncccc23)c1]

¹H NMR (400 MHz, DMSO) & 8.30 (t, J=7.3 Hz, 2H), 7.82 (s, 1H), 7.33 (s, 1H), 7.28-7.17 (m, 2H), 7.16-7.03 (m, 3H), 6.95 (s, 1H), 6.83 (d, J=7.5 Hz, 1H), 6.49 (d, J=7.9 Hz, 1H), 5.59 (s, 2H), 5.13 (s, 2H). ¹³C NMR (100 MHz, DMSO) δ 160.36 (d, J=245.3 Hz), 149.62, 148.03, 143.45, 135.26, 130.13 (d, J=10.9 Hz), 130.113, 129.88, 128.66, 126.39, 125.42 (d, J=15.0 Hz), 125.11, 118.26, 116.80, 115.87 (d, J=21.0 Hz), 115.41, 114.58, 112.43, 112.33, 41.64. HRMS m/z (ESI) calculated for C₂₀H₁₆FN₃, 318.1406, [M+H]⁺, found: 318.1398.

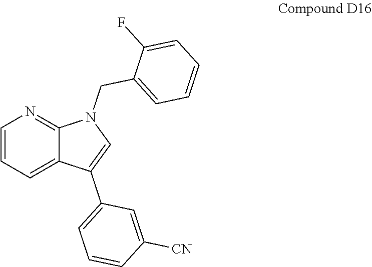 [SMILES: N#Cc1cccc(-c2cn(Cc3ccccc3F)c3ncccc23)c1]

¹H NMR (400 MHz, DMSO) δ 8.57-8.31 (m, 2H), 8.30-8.12 (m, 2H), 8.12-7.94 (m, 1H), 7.83-7.52 (m, 2H), 7.46-6.99 (m, 5H), 5.61 (s, 2H). ¹³C NMR (100 MHz, DMSO) δ 160.37 (d, J=245.4 Hz), 148.05, 143.91, 136.23, 131.10 (d, J=3.5 Hz), 130.58 (d, J=4.6 Hz), 130.26 (d, J=3.3 Hz), 130.20, 129.75, 128.67 (d, J=4.3 Hz), 128.37 (d, J=4.2 Hz), 125.15, 125.08 (d, J=2.5 Hz), 125.00, 119.39, 117.73, 117.39, 115.89 (d, J=20.9 Hz), 112.61, 112.57, 41.81 (d, J=4.1 Hz). HRMS m/z (ESI) calculated for C₂₁H₁₄FN₃, 328.125, [M+H]⁺, found: 328.1239.

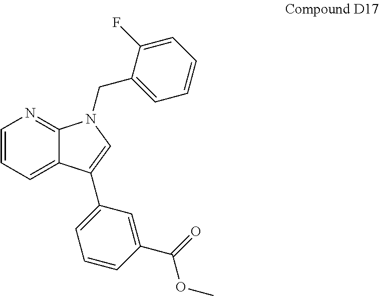 [SMILES: COC(=O)c1cccc(-c2cn(Cc3ccccc3F)c3ncccc23)c1]

¹H NMR (400 MHz, DMSO) δ 8.35 (dd, J=11.9, 6.2 Hz, 2H), 8.25 (s, 1H), 8.15 (s, 1H), 8.02 (d, J=7.8 Hz, 1H), 7.87 (d, J=7.7 Hz, 1H), 7.62 (d, J=7.7 Hz, 1H), 7.34 (d, J=5.1 Hz, 1H), 7.28-7.20 (m, 2H), 7.13 (d, J=4.7 Hz, 2H), 5.66 (d, J=29.4 Hz, 2H), 3.90 (s, 3H). 13C NMR (100 MHz, DMSO) δ 162.94 (d, J=769.3 Hz), 148.08, 143.84, 135.45, 131.39, 130.85, 130.21, 130.10 (d, J=5.7 Hz), 130.01, 128.34, 127.86, 127.03, 126.98, 125.26 (d, J=14.9 Hz), 125.13, 117.95, 117.32, 115.98, 115.77, 113.46, 52.72, 41.81 (d, J=4.1 Hz). HRMS m/z (ESI) calculated for C₂₂H₁₇FN₂O₂, 361.1352, [M+H]⁺, found: 361.1218.

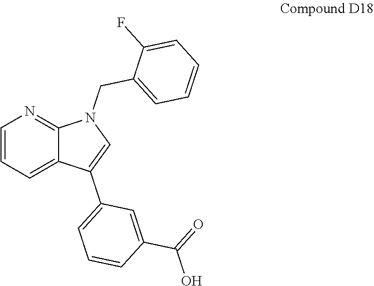 [SMILES: O=C(O)c1cccc(-c2cn(Cc3ccccc3F)c3ncccc23)c1]

¹H NMR (400 MHz, DMSO) δ 13.07 (s, 1H), 8.34 (dd, J=14.3, 6.2 Hz, 2H), 8.27 (s, 1H), 8.13 (d, J=2.0 Hz, 1H), 7.98 (d, J=7.5 Hz, 1H), 7.86 (d, J=7.7 Hz, 1H), 7.59 (t, J=7.6 Hz, 1H), 7.33 (dd, J=7.6, 3.4 Hz, 1H), 7.29-7.17 (m, 2H), 7.13 (d, J=4.8 Hz, 2H), 5.63 (s, 2H). 13C NMR (100 MHz, DMSO) δ 167.85, 160.34 (d, J=245.3 Hz), 148.08, 143.80, 135.25, 131.97, 130.96, 130.15 (d, J=9.4 Hz), 130.06, 129.80, 128.33, 127.70, 127.22, 125.27 (d, J=14.8 Hz), 125.10 (d, J=3.3 Hz), 117.98, 117.27, 115.97, 115.76, 113.65, 41.81 (d, J=4.2 Hz). HRMS m/z (ESI) calculated for C₂₁H₁₅FN₂O₂, 347.1196, [M+H]⁺, found: 347.1183.

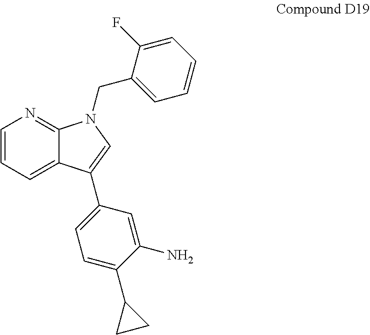 [SMILES: Nc1cc(-c2cn(Cc3ccccc3F)c3ncccc23)ccc1C1CC1]

¹H NMR (400 MHz, DMSO) δ 8.29 (t, J=6.5 Hz, 2H), 7.77 (s, 1H), 7.33 (s, 1H), 7.28-7.16 (m, 2H), 7.11 (d, J=5.7 Hz, 2H), 7.01 (s, 1H), 6.86 (d, J=7.8 Hz, 1H), 6.79 (d, J=7.1 Hz, 1H), 5.58 (s, 2H), 5.03 (s, 2H), 1.69 (s, 1H), 0.86 (d, J=7.6 Hz, 2H), 0.50 (d, J=4.4 Hz, 2H). 13C NMR (100 MHz, DMSO) δ 160.36 (d, J=245.5 Hz), 148.52, 148.01, 143.41, 132.91, 130.18, 130.10, 128.67, 127.48, 126.08, 125.10 (d, J=2.9 Hz), 124.30, 118.28, 116.73, 115.98, 115.77, 115.34, 114.68, 112.12, 41.64 (d, J=4.5 Hz), 11.27, 6.18 (2C). HRMS m/z (ESI) calculated for C₂₃H₂₀FN₃, 358.1719, [M+H]⁺, found: 358.1711.

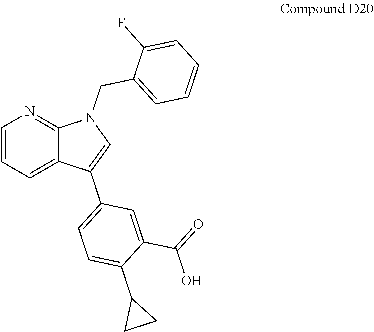 [SMILES: O=C(O)c1cc(-c2cn(Cc3ccccc3F)c3ncccc23)ccc1C1CC1]

¹H NMR (400 MHz, DMSO) δ 12.96 (s, 1H), 8.38-8.31 (m, 1H), 8.28 (d, J=7.9 Hz, 1H), 8.03 (d, J=12.2 Hz, 2H), 7.76 (d, J=8.1 Hz, 1H), 7.34 (d, J=6.0 Hz, 1H), 7.23 (t, J=7.3 Hz, 2H), 7.09 (dd, J=18.7, 6.3 Hz, 3H), 5.61 (s, 2H), 2.69 (d, J=3.5 Hz, 1H), 0.99 (d, J=8.1 Hz, 2H), 0.72 (d, J=4.8 Hz, 2H). ¹³C NMR (100 MHz, DMSO) & 169.79, 160.32 (d, J=245.0 Hz), 148.03, 143.69, 141.71, 133.11, 131.79, 130.13 (d, J=8.2 Hz), 130.04 (d, J=4.0 Hz), 129.64, 128.32, 127.40, 127.22, 125.87, 125.31 (d, J=14.9 Hz), 125.09 (d, J=3.2 Hz), 118.01, 117.14, 115.86 (d, J=21.0 Hz), 113.57, 41.75 (d, J=4.4 Hz), 12.98, 9.68 (2C). HRMS m/z (ESI) calculated for C₂₄H₁₉FN₂O₂, 387.1509, [M+H], found: 387.1498.

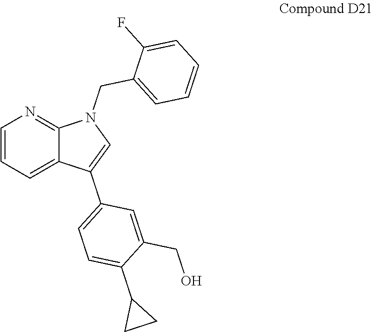 [SMILES: OCc1cc(-c2cn(Cc3ccccc3F)c3ncccc23)ccc1C1CC1]

¹H NMR (400 MHz, DMSO) δ 8.32 (s, 2H), 7.91 (s, 1H), 7.73 (s, 1H), 7.47 (d, J=7.6 Hz, 1H), 7.34 (d, J=4.9 Hz, 1H), 7.23 (t, J=8.8 Hz, 2H), 7.11 (s, 2H), 7.02 (d, J=7.7 Hz, 1H), 5.60 (s, 2H), 5.17 (d, J=5.2 Hz, 1H), 4.75 (d, J=5.2 Hz, 2H), 1.97 (s, 1H), 0.91 (d, J=7.1 Hz, 2H), 0.63 (d, J=3.5 Hz, 2H). ¹³C NMR (100 MHz, DMSO) δ 160.35 (d, J=245.2 Hz), 148.06, 143.54, 142.28, 138.03, 131.95, 130.13 (d, J=8.2 Hz), 130.05 (d, J=4.2 Hz), 128.56, 126.49, 125.68, 125.42 (d, J=14.6 Hz), 125.09 (d, J=3.5 Hz), 124.94, 124.69, 118.22, 116.92, 115.87 (d, J=21.0 Hz), 114.87, 61.15, 41.70 (d, J=4.3 Hz), 19.03, 12.03, 7.50 (2C). HRMS m/z (ESI) calculated for C₂₄H₂₁FN₂O, 373.1716, [M+H]⁺, found: 373.1707.

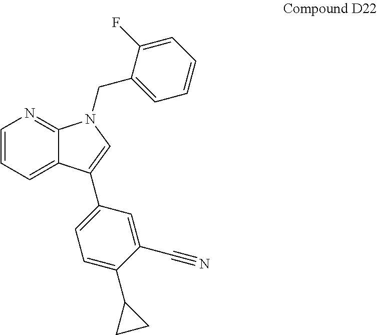 [SMILES: N#Cc1cc(-c2cn(Cc3ccccc3F)c3ncccc23)ccc1C1CC1]

¹H NMR (400 MHz, DMSO) δ 8.43-8.30 (m, 2H), 8.13 (s, 1H), 8.06 (s, 1H), 7.91 (d, J=8.3 Hz, 1H), 7.34 (d, J=6.7 Hz, 1H), 7.24 (dd, J=11.2, 6.2 Hz, 2H), 7.14 (p, J=7.6 Hz, 3H), 5.60 (s, 2H), 2.28-2.09 (m, 1H), 1.14 (t, J=7.0 Hz, 2H), 0.85 (d, J=4.8 Hz, 2H). ¹³C NMR (100 MHz, DMSO) & 160.36 (d, J=245.4 Hz), 148.00, 144.86, 143.81, 133.08, 131.36, 130.23 (d, J=4.3 Hz), 130.17, 129.98, 128.58, 127.88, 125.59, 125.15 (d, J=10.3 Hz), 125.06, 118.67, 117.75, 117.27, 115.88 (d, J=21.0 Hz), 112.92, 112.49, 41.76 (d, J=4.3 Hz), 14.09, 9.89 (2C). HRMS m/z (ESI) calculated for C₂₄H₁₈FN₃, 368.1563, [M+H]⁺, found: 368.1554.

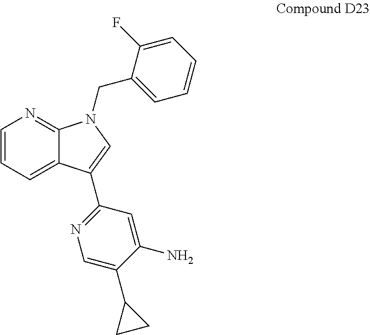 [SMILES: Nc1cc(-c2cn(Cc3ccccc3F)c3ncccc23)ncc1C1CC1]

¹H NMR (400 MHz, DMSO) δ 8.56 (d, J=8.0 Hz, 1H), 8.37 (d, J=4.5 Hz, 1H), 8.22 (s, 1H), 7.83 (s, 1H), 7.43-7.05 (m, 6H), 6.75 (s, 2H), 5.62 (s, 2H), 1.65 (s, 1H), 0.92 (d, J=8.1 Hz, 2H), 0.59 (d, J=5.1 Hz, 2H). ¹³C NMR (100 MHz, DMSO) δ 160.44 (d, J=245.2 Hz), 154.47, 151.92, 148.08, 147.75, 143.43, 130.30, 130.23 (d, J=5.9 Hz), 127.72, 125.38, 125.23, 125.11 (d, J=3.5 Hz), 119.14, 118.46, 116.99, 116.01, 115.80, 115.19, 103.82, 8.99, 5.53 (2C). HRMS m/z (ESI) calculated for C₂₂H₁₉FN₄, 359.1672, [M+H]⁺, found: 359.1659.

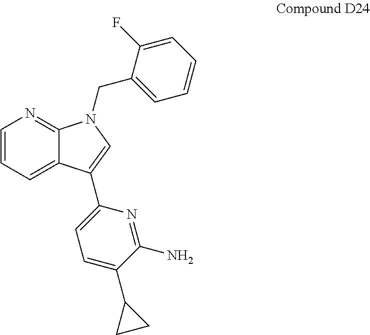 [SMILES: Nc1nc(-c2cn(Cc3ccccc3F)c3ncccc23)ccc1C1CC1]

¹H NMR (400 MHz, DMSO) δ 8.90 (dd, J=7.9, 1.3 Hz, 1H), 8.32 (dd, J=4.5, 1.2 Hz, 1H), 8.10 (s, 1H), 7.38-7.27 (m, 1H), 7.28-7.16 (m, 2H), 7.18-7.04 (m, 3H), 6.97 (d, J=7.6 Hz, 1H), 5.87 (s, 2H), 5.61 (s, 2H), 1.80-1.55 (m, 1H), 0.99-0.65 (m, 2H), 0.61-0.39 (m, 2H). 13C NMR (101 MHz, DMSO) δ 160.38 (d, J=245.3 Hz), 159.01, 150.05, 148.16, 143.48, 134.69, 131.22, 130.18, 130.07 (d, J=4.2 Hz), 127.88, 125.35 (d, J=14.9 Hz), 125.06 (d, J=3.4 Hz), 118.65, 118.05, 117.01, 115.86 (d, J=21.0 Hz), 114.97, 108.36, 41.74 (d, J=3.9 Hz), 11.09, 6.24 (2C). HRMS m/z (ESI) calculated for C₂₂H₁₉FN₄, 358.1594, [M+H]⁺, found: 359.1662.

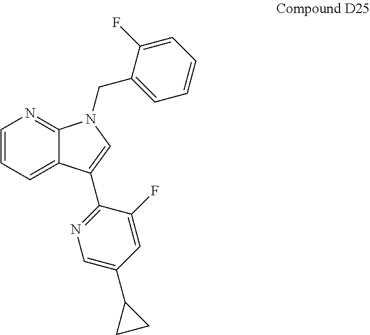 [SMILES: Fc1ccccc1Cn1cc(-c2ncc(C3CC3)cc2F)c2cccnc21]

¹H NMR (400 MHz, DMSO) δ 8.88 (d, J=7.9 Hz, 1H), 8.38 (dd, J=8.4, 2.9 Hz, 2H), 8.19 (s, 1H), 7.48-7.30 (m, 2H), 7.29-7.21 (m, 2H), 7.16-7.08 (m, 2H), 5.67 (s, 2H), 2.03 (s, 1H), 1.08-0.99 (m, 2H), 0.90-0.71 (m, 2H). ¹³C NMR (100 MHz, DMSO) & 160.40 (d, J=245.1 Hz), 156.50 (d, J=257.3 Hz), 147.61, 144.14, 143.73 (d, J=3.6 Hz), 139.98 (d, J=12.0 Hz), 138.91 (d, J=2.7 Hz), 131.42, 130.45 (d, J=17.5 Hz), 130.19 (d, J=8.2 Hz), 130.03 (d, J=4.0 Hz), 125.17 (d, J=18.0 Hz), 119.71 (d, J=19.9 Hz), 118.88, 117.51, 115.99, 115.78, 109.03 (d, J=6.5 Hz), 41.98, 12.72, 10.11 (2C). HRMS m/z (ESI) calculated for C₂₂H₁₇F₂N₃, 362.1469, [M+H]⁺, found: 362.1460.

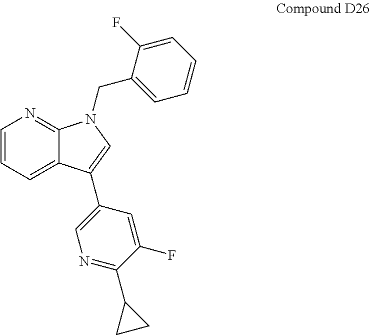 [SMILES: Fc1ccccc1Cn1cc(-c2cnc(C3CC3)c(F)c2)c2cccnc21]

¹H NMR (400 MHz, DMSO) δ 8.68 (s, 1H), 8.37 (dd, J=14.8, 6.0 Hz, 2H), 8.17 (s, 1H), 7.93 (d, J=11.5 Hz, 1H), 7.40-7.30 (m, 1H), 7.28-7.17 (m, 2H), 7.13 (d, J=4.7 Hz, 2H), 5.60 (s, 2H), 2.38-2.23 (m, 1H), 1.03 (d, J=6.1 Hz, 4H). ¹³C NMR (100 MHz, DMSO) δ 160.34 (d, J=245.4 Hz), 158.03 (d, J=253.2 Hz), 147.98, 147.17 (d, J=14.6 Hz), 143.94, 142.74 (d, J=4.4 Hz), 130.23 (d, J=8.3 Hz), 130.13 (d, J=3.8 Hz), 129.45 (d, J=4.0 Hz), 128.80, 128.20, 125.14 (d, J=2.8 Hz), 125.05 (d, J=8.2 Hz), 119.66 (d, J=19.7 Hz), 117.81, 117.38, 116.00, 115.90 (d, J=21.0 Hz), 41.85, 10.32, 9.38 (2C). HRMS m/z (ESI) calculated for C₂₂H₁₇F₂N₃, 362.1469, [M+H]⁺, found: 362.1459.

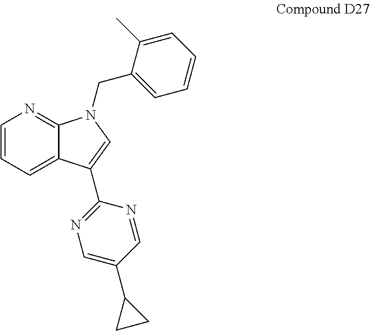 [SMILES: Cc1ccccc1Cn1cc(-c2ncc(C3CC3)cn2)c2cccnc21]

¹H NMR (400 MHz, DMSO) δ 8.81 (d, J=7.9 Hz, 1H), 8.55 (d, J=0.8 Hz, 2H), 8.35 (d, J=4.6 Hz, 1H), 8.23 (s, 1H), 7.28 (dd, J=4.9, 3.0 Hz, 1H), 7.21 (d, J=7.3 Hz, 2H), 7.13 (d, J=7.1 Hz, 1H), 6.91 (d, J=7.5 Hz, 1H), 5.57 (s, 2H), 2.33 (s, 3H), 2.08-1.76 (m, 1H), 1.02 (dd, J=5.4, 2.9 Hz, 2H), 0.84 (d, J=4.9 Hz, 2H). ¹³C NMR (100 MHz, DMSO) δ 160.56, 155.02 (2C), 148.42, 143.86, 136.20, 136.14, 133.03, 131.41, 130.89, 130.75, 128.05, 126.59, 118.66, 117.73, 113.50, 45.88, 19.22, 11.01, 9.22 (2C). HRMS m/z (ESI) calculated for C₂₂H₂₀N₄, 341.1766, [M+H]⁺, found: 341.1757.

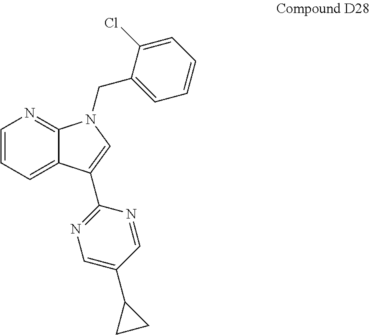 [SMILES: Clc1ccccc1Cn1cc(-c2ncc(C3CC3)cn2)c2cccnc21]

¹H NMR (400 MHz, DMSO) δ 8.82 (d, J=7.8 Hz, 1H), 8.57 (s, 2H), 8.47-8.21 (m, 2H), 7.53 (d, J=7.8 Hz, 1H), 7.29 (ddd, J=21.5, 17.0, 7.1 Hz, 3H), 6.92 (d, J=7.5 Hz, 1H), 5.68 (s, 2H), 1.93 (d, J=4.9 Hz, 1H), 1.11-0.92 (m, 2H), 0.92-0.76 (m, 2H). ¹³C NMR (100 MHz, DMSO) & 160.51, 155.02 (2C), 148.33, 143.99, 135.49, 133.13, 132.40, 131.67, 130.97, 129.95, 129.85, 129.38, 128.02, 118.77, 117.85, 113.72, 45.86, 11.03, 9.25 (2C). HRMS m/z (ESI) calculated for C₂₁H₁₇ClN₄, 361.1220, [M+H]⁺, found: 361.1209. (d, J=8.1 Hz, 2H), 0.93-0.71 (m, 2H). ¹³C NMR (100 MHz, DMSO) § 160.61, 155.03 (2C), 148.28, 143.87, 138.38, 133.02, 131.73, 130.83, 129.08 (2C), 128.08 (2C), 128.00, 118.81, 117.68, 113.41, 47.92, 11.02, 9.22 (2C). HRMS m/z (ESI) calculated for C₂₁H₁₈N₄, 327.1609, [M+H]⁺, found: 327.1598.

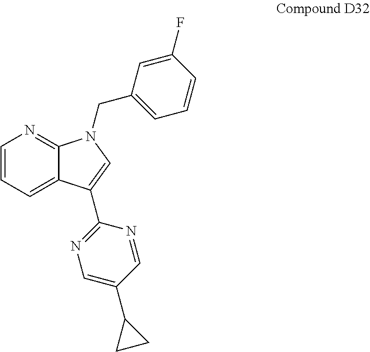 [SMILES: Fc1cccc(Cn2cc(-c3ncc(C4CC4)cn3)c3cccnc32)c1]

¹H NMR (400 MHz, DMSO) δ 8.81 (d, J=7.9 Hz, 1H), 8.58 (s, 2H), 8.53 (s, 1H), 8.38 (d, J=3.3 Hz, 1H), 7.43-7.33 (m, 1H), 7.29 (dd, J=7.2, 4.8 Hz, 1H), 7.19 (t, J=10.5 Hz, 2H), 7.11 (t, J=9.0 Hz, 1H), 5.61 (s, 2H), 1.94 (dd, J=8.4, 3.6 Hz, 1H), 1.03 (d, J=8.3 Hz, 2H), 0.86 (d, J=4.9 Hz, 2H).

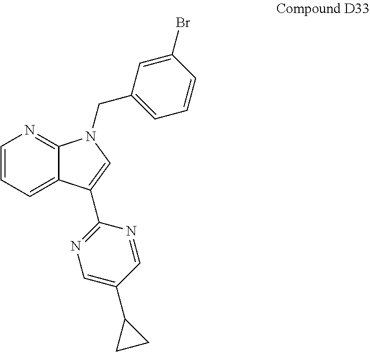 [SMILES: Brc1cccc(Cn2cc(-c3ncc(C4CC4)cn3)c3cccnc32)c1]

¹H NMR (400 MHz, DMSO) § 8.80 (dd, J=7.9, 1.6 Hz, 1H), 8.57 (s, 2H), 8.53 (s, 1H), 8.37 (dd, J=4.7, 1.6 Hz, 1H), 7.59 (d, J=1.6 Hz, 1H), 7.49-7.43 (m, 1H), 7.30 (ddd, J=12.1, 7.8, 6.3 Hz, 3H), 5.58 (s, 2H), 1.95 (tt, J=8.5, 5.1 Hz, 1H), 1.03 (ddd, J=8.4, 6.5, 4.3 Hz, 2H), 0.91-0.78 (m, 2H).

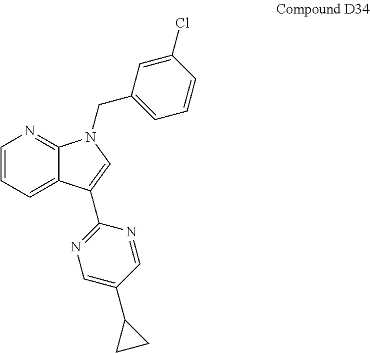 [SMILES: Clc1cccc(Cn2cc(-c3ncc(C4CC4)cn3)c3cccnc32)c1]

¹H NMR (400 MHz, DMSO) δ 8.83-8.76 (m, 1H), 8.57 (s, 2H), 8.52 (s, 1H), 8.40-8.32 (m, 1H), 7.44 (s, 1H), 7.35 (d, J=6.3 Hz, 2H), 7.28 (dd, J=7.8, 4.7 Hz, 2H), 5.59 (s, 2H), 1.94 (d, J=4.9 Hz, 1H), 1.11-0.97 (m, 2H), 0.92-0.76 (m, 2H). ¹³C NMR (100 MHz, DMSO) δ 160.54, 155.04 (2C), 148.18, 143.98, 140.88, 133.61, 133.12, 131.76, 130.98 (d, J=10.8 Hz), 128.00, 127.89, 126.75, 118.83, 117.81, 113.60, 47.36, 11.02, 9.24 (2C). HRMS m/z (ESI) calculated for C₂₁H₁₇ClN₄, 361.1220, [M+H]⁺, found: 361.1210.

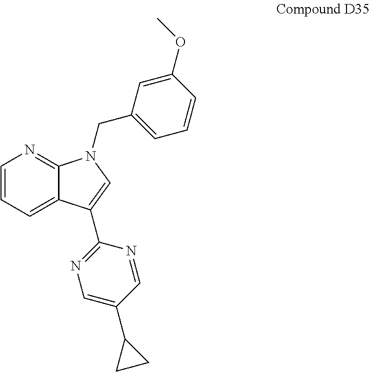 [SMILES: COc1cccc(Cn2cc(-c3ncc(C4CC4)cn3)c3cccnc32)c1]

¹H NMR (400 MHz, DMSO) § 8.79 (dd, J=7.9, 1.3 Hz, 1H), 8.56 (s, 2H), 8.46 (s, 1H), 8.37 (dd, J=4.5, 1.2 Hz, 1H), 7.39-7.12 (m, 2H), 6.96 (s, 1H), 6.93-6.74 (m, 2H), 5.55 (s, 2H), 3.71 (s, 3H), 2.07-1.81 (m, 1H), 1.03 (qd, J=6.2, 2.8 Hz, 2H), 0.85 (q, J=4.8 Hz, 2H).

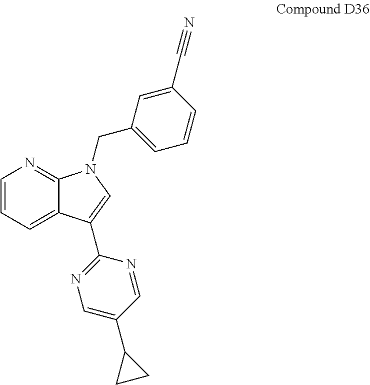 [SMILES: N#Cc1cccc(Cn2cc(-c3ncc(C4CC4)cn3)c3cccnc32)c1]

H NMR (400 MHz, DMSO) δ 8.80 (t, J=8.2 Hz, 1H), 8.59 (d, J=10.3 Hz, 2H), 8.55 (s, 1H), 8.35 (t, J=8.5 Hz, 1H), 7.86 (s, 1H), 7.76 (d, J=7.6 Hz, 1H), 7.65 (d, J=7.8 Hz, 1H), 7.54 (t, J=7.7 Hz, 1H), 7.33-7.23 (m, 1H), 1.97 (tdd, J=13.4, 8.5, 5.1 Hz, 1H), 1.13-0.98 (m, 2H), 0.91-0.79 (m, 2H).

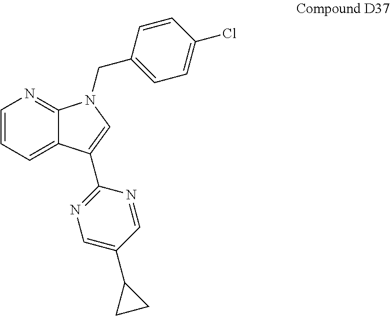 [SMILES: Clc1ccc(Cn2cc(-c3ncc(C4CC4)cn3)c3cccnc32)cc1]

¹H NMR (400 MHz, DMSO) § 8.80 (d, J=7.9 Hz, 1H), 8.57 (d, J=2.2 Hz, 2H), 8.50 (d, J=1.6 Hz, 1H), 8.41-8.31 (m, 1H), 7.38 (s, 4H), 7.33-7.23 (m, 1H), 5.59 (s, 2H), 1.94 (d, J=3.2 Hz, 1H), 1.11-0.98 (m, 2H), 0.91-0.79 (m, 2H).

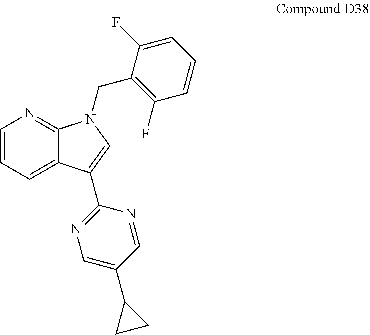 [SMILES: Fc1cccc(F)c1Cn1cc(-c2ncc(C3CC3)cn2)c2cccnc21]

¹H NMR (400 MHz, DMSO) δ 8.76 (d, J=7.9 Hz, 1H), 8.56 (s, 2H), 8.36 (d, J=4.6 Hz, 1H), 8.23 (s, 1H), 7.55-7.40 (m, 1H), 7.34-7.23 (m, 1H), 7.16 (t, J=7.9 Hz, 2H), 5.66 (s, 2H), 2.03-1.85 (m, 1H), 1.08-0.94 (m, 2H), 0.85 (t, J=4.7 Hz, 2H). ¹³C NMR (100 MHz, DMSO) δ 162.70 (d, J=7.8 Hz), 160.38, 160.23 (d, J=7.8 Hz), 155.04 (2C), 148.15, 143.97, 133.16, 131.47 (t, J=10.3 Hz), 131.00, 130.79, 118.55, 117.80, 113.68, 113.13 (t, J=19.1 Hz), 112.49 (d, J=6.1 Hz), 112.30 (d, J=6.0 Hz), 36.22, 11.01, 9.24 (2C). HRMS m/z (ESI) calculated for C₂₁H₁₆F₂N₄, 363.1421, [M+H]⁺, found: 393.1409.

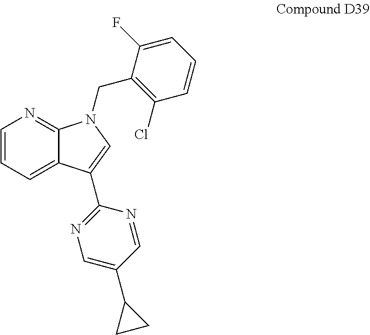 [SMILES: Fc1cccc(Cl)c1Cn1cc(-c2ncc(C3CC3)cn2)c2cccnc21]

¹H NMR (400 MHz, DMSO) δ 8.90-8.67 (m, 1H), 8.54 (d, J=1.9 Hz, 2H), 8.45-8.27 (m, 1H), 8.05 (s, 1H), 7.55-7.39 (m, 2H), 7.39-7.21 (m, 2H), 5.70 (s, 2H), 2.00-1.78 (m, 1H), 1.09-0.91 (m, 2H), 0.91-0.69 (m, 2H).

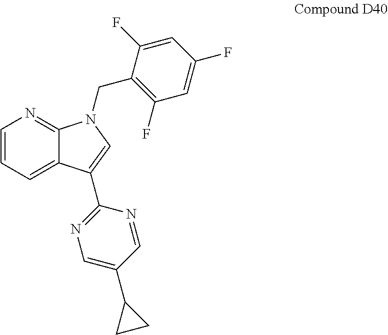 [SMILES: Fc1cc(F)c(Cn2cc(-c3ncc(C4CC4)cn3)c3cccnc32)c(F)c1]

¹H NMR (400 MHz, DMSO) δ 8.76 (d, J=7.9 Hz, 1H), 8.55 (d, J=9.4 Hz, 2H), 8.41-8.30 (m, 1H), 8.26 (s, 1H), 7.37-7.15 (m, 3H), 5.61 (s, 2H), 2.00-1.83 (m, 1H), 1.03 (d, J=8.1 Hz, 2H), 0.92-0.68 (m, 2H).

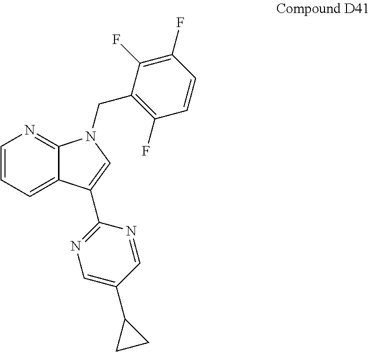 [SMILES: Fc1ccc(F)c(Cn2cc(-c3ncc(C4CC4)cn3)c3cccnc32)c1F]

¹H NMR (400 MHz, DMSO) δ 8.77 (d, J=7.9 Hz, 1H), 8.56 (d, J=5.2 Hz, 2H), 8.39-8.34 (m, 1H), 8.32 (s, 1H), 7.59-7.43 (m, 1H), 7.27 (dd, J=7.9, 4.7 Hz, 1H), 7.23-7.12 (m, 1H), 5.70 (s, 2H), 1.99-1.89 (m, 1H), 1.10-0.99 (m, 2H), 0.90-0.79 (m, 2H).

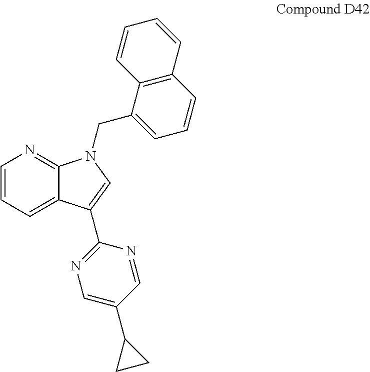 [SMILES: c1ccc2c(Cn3cc(-c4ncc(C5CC5)cn4)c4cccnc43)cccc2c1]

¹H NMR (400 MHz, DMSO) δ 8.82 (d, J=7.9 Hz, 1H), 8.53 (s, 2H), 8.42 (d, J=2.6 Hz, 1H), 8.31-8.21 (m, 2H), 8.02-7.94 (m, 1H), 7.91 (d, J=8.2 Hz, 1H), 7.62-7.51 (m, 2H), 7.46 (t, J=7.7 Hz, 1H), 7.34-7.23 (m, 2H), 6.06 (s, 2H), 1.90 (d, J=5.0 Hz, 1H), 1.04-0.96 (m, 2H), 0.85-0.79 (m, 2H).

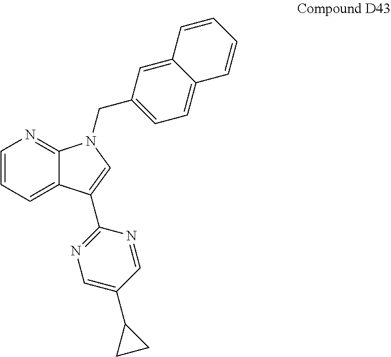 [SMILES: c1ccc2cc(Cn3cc(-c4ncc(C5CC5)cn4)c4cccnc43)ccc2c1]

¹H NMR (400 MHz, DMSO) δ 8.81 (d, J=7.9 Hz, 1H), 8.56 (d, J=0.8 Hz, 2H), 8.51 (s, 1H), 8.38 (d, J=4.6 Hz, 1H), 8.00-7.79 (m, 4H), 7.50 (dd, J=11.0, 6.5 Hz, 3H), 7.29 (ddd, J=7.8, 4.7, 1.0 Hz, 1H), 5.75 (s, 2H), 1.93 (s, 1H), 1.02 (d, J=8.1 Hz, 2H), 0.84 (d, J=4.9 Hz, 2H).

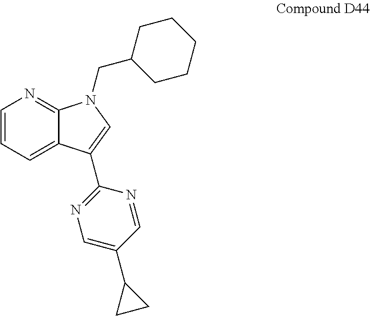 [SMILES: c1cnc2c(c1)c(-c1ncc(C3CC3)cn1)cn2CC1CCCCC1]

¹H NMR (400 MHz, DMSO) δ 8.77 (d, J=7.6 Hz, 1H), 8.57 (s, 2H), 8.33 (s, 2H), 7.24 (dd, J=6.9, 4.8 Hz, 1H), 4.20 (d, J=6.9 Hz, 2H), 1.95 (s, 2H), 1.56 (dd, J=39.2, 17.9 Hz, 5H), 1.20-0.95 (m, 7H), 0.85 (d, J=4.3 Hz, 2H).

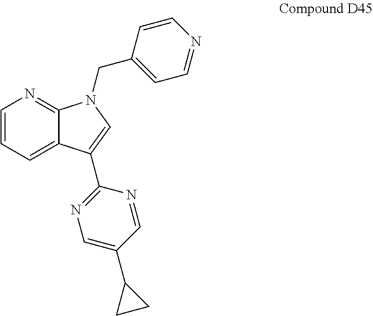 [SMILES: c1cnc2c(c1)c(-c1ncc(C3CC3)cn1)cn2Cc1ccncc1]

¹H NMR (400 MHz, DMSO) § 8.82 (d, J=7.9 Hz, 1H), 8.59 (s, 2H), 8.54 (s, 1H), 8.51 (d, J=4.7 Hz, 2H), 8.34 (d, J=4.5 Hz, 1H), 7.29 (dd, J=7.8, 4.7 Hz, 1H), 7.21 (d, J=4.9 Hz, 2H), 5.65 (s, 2H), 1.95 (dd, J=8.6, 3.8 Hz, 1H), 1.14-0.95 (m, 2H), 0.86 (q, J=5.0 Hz, 2H).

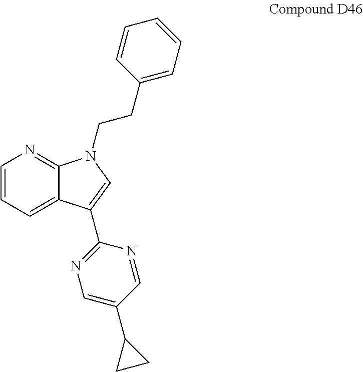 [SMILES: c1ccc(CCn2cc(-c3ncc(C4CC4)cn3)c3cccnc32)cc1]

¹H NMR (400 MHz, DMSO) δ 8.86-8.71 (m, 1H), 8.55 (s, 2H), 8.43-8.25 (m, 2H), 7.41-7.05 (m, 6H), 4.60 (t, J=7.2 Hz, 2H), 3.20 (t, J=7.2 Hz, 2H), 2.05-1.74 (m, 1H), 1.02 (d, J=8.2 Hz, 2H), 0.85 (d, J=5.0 Hz, 2H).

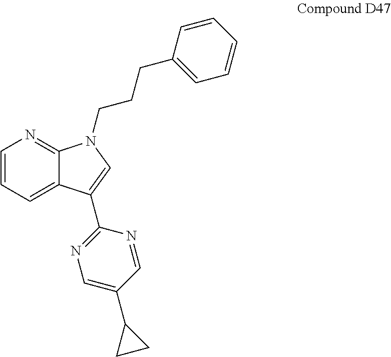 [SMILES: c1ccc(CCCn2cc(-c3ncc(C4CC4)cn3)c3cccnc32)cc1]

¹H NMR (400 MHz, DMSO) δ 8.78 (d, J=7.8 Hz, 1H), 8.57 (s, 2H), 8.42 (s, 1H), 8.34 (d, J=4.3 Hz, 1H), 7.32-7.23 (m, 3H), 7.22-7.14 (m, 4H), 4.39 (t, J=7.0 Hz, 2H), 2.65-2.57 (m, 2H), 2.29-2.12 (m, 2H), 2.05-1.89 (m, 1H), 1.03 (d, J=6.6 Hz, 2H), 0.86 (d, J=5.2 Hz, 2H).

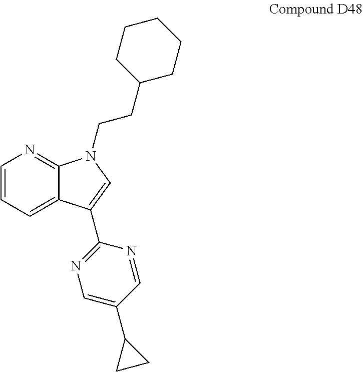 [SMILES: c1cnc2c(c1)c(-c1ncc(C3CC3)cn1)cn2CCC1CCCCC1]

¹H NMR (400 MHz, DMSO) δ 8.76 (dd, J=7.9, 1.5 Hz, 1H), 8.56 (s, 2H), 8.38 (s, 1H), 8.33 (dd, J=4.6, 1.5 Hz, 1H), 7.24 (dd, J=7.9, 4.7 Hz, 1H), 4.37 (t, J=7.3 Hz, 2H), 1.94 (s, 1H), 1.75 (dd, J=14.3, 6.5 Hz, 4H), 1.70-1.48 (m, 3H), 1.11 (d, J=7.6 Hz, 4H), 1.07-0.98 (m, 2H), 0.95 (s, 2H), 0.85 (dd, J=5.0, 1.8 Hz, 2H).

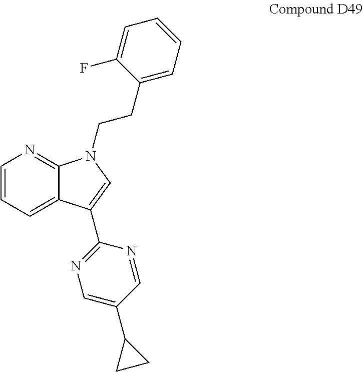 [SMILES: Fc1ccccc1CCn1cc(-c2ncc(C3CC3)cn2)c2cccnc21]

¹H NMR (400 MHz, DMSO) δ 8.75 (dd, J=7.9, 1.4 Hz, 1H), 8.55 (s, 2H), 8.33 (dd, J=4.6, 1.3 Hz, 1H), 8.28 (s, 1H), 7.28-7.18 (m, 3H), 7.13 (d, J=9.9 Hz, 1H), 7.05 (t, J=7.4 Hz, 1H), 4.61 (t, J=7.2 Hz, 2H), 3.24 (t, J=7.1 Hz, 2H), 2.05-1.84 (m, 1H), 1.15-0.97 (m, 2H), 0.93-0.75 (m, 2H).

### Compound D50

### 1-(2-chlorophenethyl)-3-(5-cyclopropylpyrimidin-2-yl)-1H-pyrrolo[2,3-b]pyridine (s32)

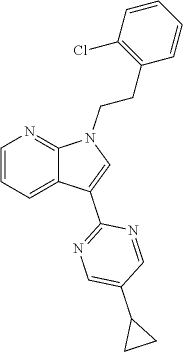 [SMILES: Clc1ccccc1CCn1cc(-c2ncc(C3CC3)cn2)c2cccnc21]

¹H NMR (400 MHz, DMSO) δ 8.75 (dd, J=7.9, 1.6 Hz, 1H), 8.55 (s, 2H), 8.33 (dd, J=4.6, 1.5 Hz, 1H), 8.26 (s, 1H), 7.44-7.38 (m, 1H), 7.23 (ddd, J=6.5, 3.7, 1.8 Hz, 4H), 4.62 (t, J=7.2 Hz, 2H), 3.31 (d, J=7.1 Hz, 2H), 1.99-1.87 (m, 1H), 1.11-0.98 (m, 2H), 0.90-0.76 (m, 2H).

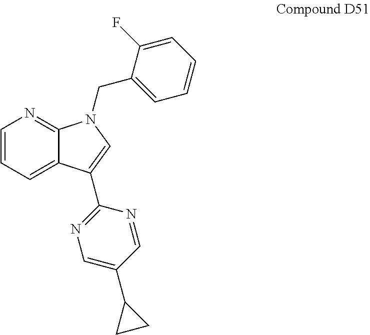 [SMILES: Fc1ccccc1Cn1cc(-c2ncc(C3CC3)cn2)c2cccnc21]

¹H NMR (400 MHz, DMSO) δ 8.76 (d, J=7.9 Hz, 1H), 8.55 (s, 2H), 8.35 (d, J=5.6 Hz, 2H), 7.36-7.19 (m, 2H), 7.09 (d, J=10.1 Hz, 1H), 7.06-6.94 (m, 2H), 4.62 (t, J=7.2 Hz, 2H), 3.23 (t, J=7.2 Hz, 2H), 2.00-1.83 (m, 1H), 1.11-0.96 (m, 2H), 0.85 (q, J=4.9 Hz, 2H).

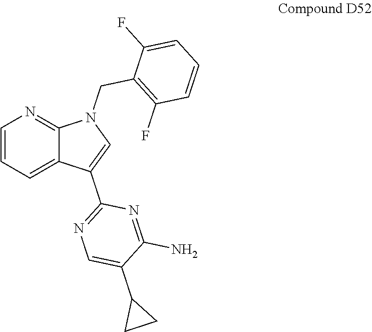 [SMILES: Nc1nc(-c2cn(Cc3c(F)cccc3F)c3ncccc23)ncc1C1CC1]

¹H NMR (400 MHz, DMSO) δ 9.04-8.80 (m, 1H), 8.35 (dd, J=3.1, 1.5 Hz, 1H), 8.05 (s, 1H), 7.90 (s, 1H), 7.54-7.39 (m, 1H), 7.29-7.07 (m, 3H), 6.78 (s, 2H), 5.63 (s, 2H), 1.63 (dd, J=8.5, 3.6 Hz, 1H), 0.94-0.73 (m, 2H), 0.57 (d, J=5.1 Hz, 2H).

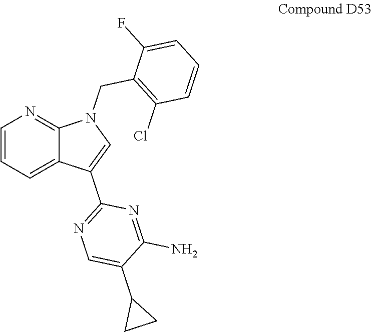 [SMILES: Nc1nc(-c2cn(Cc3c(F)cccc3Cl)c3ncccc23)ncc1C1CC1]

¹H NMR (400 MHz, DMSO) δ 8.91 (d, J=7.9 Hz, 1H), 8.45-8.28 (m, 1H), 7.87 (d, J=11.4 Hz, 2H), 7.58-7.39 (m, 2H), 7.35 (t, J=8.9 Hz, 1H), 7.29-7.17 (m, 1H), 6.75 (s, 2H), 5.68 (s, 2H), 1.69-1.51 (m, 1H), 0.86 (d, J=8.0 Hz, 2H), 0.55 (d, J=5.0 Hz, 2H). ¹³C NMR (100 MHz, DMSO) § 163.47, 162.00 (d, J=249.8 Hz), 160.12, 152.31, 148.21, 143.63, 135.46 (d, J=5.1 Hz), 131.83 (d, J=9.9 Hz), 131.41, 129.11, 126.45 (d, J=3.2 Hz), 122.71 (d, J=17.6 Hz), 118.86, 117.43, 115.48 (d, J=22.2 Hz), 115.00, 114.70, 8.76, 5.62 (2C). HRMS m/z (ESI) calculated for C₂₁H₁₇ClFN₅, 394.1235, [M+H]⁺, found: 394.1225.

Determination of hDHODH Enzymatic Inhibitory Activity of Compounds

Assay buffer preparation: 100 mM HEPES buffer, 150 mM NaCl, 10% glycerol, and 0.1% Triton X-100 were mixed, adjusted to pH-8.0. A resulting mixture was diluted with Milli-Q water, and then stored at 4° C. A total volume of the enzyme activity detection system was 60 μL. The procedure was performed as follows: 1 μL of the small-molecule solution (with a final concentration of 1 μM) was added to a 96-well enzyme plate. 39 μL of a mixture containing the purified hDHODH protein (with a final concentration of 40 nM), 2,6-dichloroindophenol sodium salt (with a final concentration of 100 μM), coenzyme Q (CoQ, with a final concentration of 20 μM) were added thereto. The plate was protected from light and incubated at 37° C. with gentle shaking for 30 min. After the incubation was completed, 20 μL of dihydroorotate (DHO, with a final concentration of 200 μM) was added to each well. Absorbance was immediately measured using a microplate reader having the following procedure: kinetic assay: wavelength: 600 nm, total duration: 10 min, interval: every 20 s. Controls: negative control: Dimethyl sulfoxide (DMSO) (equal volume) without inhibitor; blank control: ultra-pure water (equal volume) without DHO substrate. All assays were conducted in triplicate. Data analysis was: inhibition (%)=[1−(V_{compound}−V_{blank})/(V_{control}−V_{blank})]×100%, where V=average reaction rate. The IC50 values were calculated by nonlinear regression analysis using GraphPad Prism 8.0, with the logarithm of compound concentration as the x-axis and inhibition as the y-axis. The experiment was repeated three times with triplicate wells per group.

TABLE 1
hDHODH enzymatic inhibitory activity results
of compounds in the present disclosure

| | Inhibition | IC₅₀ |
|---|---|---|
| Cpd. | (%) at 1 μM | (nM) |
| D1 | 77.2 | 281.5 ± 8.9 |
| D2 | 77.2 | 313.8 ± 15.2 |
| D3 | 49.4 | ND |
| D4 | 55.6 | ND |
| D5 | 53.4 | ND |
| D6 | 40.6 | ND |
| D7 | 41.8 | ND |
| D8 | 19.0 | ND |
| D9 | 6.8 | ND |
| D10 | 2.6 | ND |
| D11 | 48.3 | ND |
| D12 | 10.4 | ND |
| D13 | 22.0 | ND |
| D14 | 43.5 | ND |
| D15 | 4.7 | ND |
| D16 | 5.4 | ND |
| D17 | 6.6 | ND |
| D18 | 6.9 | ND |
| D19 | 33.1 | ND |
| D20 | 59.4 | ND |
| D21 | 56.7 | ND |
| D22 | 35.4 | ND |
| D23 | 20.1 | ND |
| D24 | 42.3 | ND |
| D25 | 27.2 | ND |
| D26 | 16.1 | ND |
| D27 | 73.6 | 285.8 ± 5.7 |
| D28 | 75.6 | 239.8 ± 14.1 |
| D29 | 75.8 | 324.4 ± 29.5 |
| D30 | 57.6 | ND |
| D31 | 65.7 | 490.9 ± 46.7 |
| D32 | 54.9 | 539.6 ± 25.6 |
| D33 | 61.3 | ND |
| D34 | 54.4 | 601.4 ± 56.1 |
| D35 | 54.5 | 520.3 ± 37.7 |
| D36 | 32.3 | ND |
| D37 | 43.3 | ND |
| D38 | 79.8 | 172.6 ± 3.5 |
| D39 | 81.3 | 168.3 ± 15.3 |
| D40 | 76.6 | 368.2 ± 33.8 |
| D41 | 72.9 | 273.2 ± 30.4 |
| D42 | 56.4 | 829.1 ± 81.9 |
| D43 | 35.7 | ND |
| D44 | 68.1 | ND |
| D45 | 28.3 | ND |
| D46 | 76.5 | 266.7 ± 9.7 |
| D47 | 58.5 | 429.7 ± 23.7 |
| D48 | 54.3 | 294.3 ± 5.6 |
| D49 | 71.7 | 228.3 ± 25.6 |
| D50 | 69.7 | ND |
| D51 | 45.3 | ND |
| D52 | 70.6 | 177.4 ± 14.2 |
| D53 | 77.4 | 173.4 ± 24.0 |

NOTE:
“ND” means that the IC₅₀ value was not detected.

Although the present disclosure is described in detail in conjunction with the foregoing embodiments, they are only a part of, not all of, the embodiments of the present disclosure. Other embodiments can be obtained based on these examples without creative efforts, and all of these embodiments shall fall within the scope of the present disclosure.

What is claimed is:

1. A compound having a structure shown in Formula I: 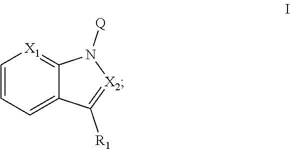
wherein
in the Formula I, Q is a structure being one selected from the group consisting of: 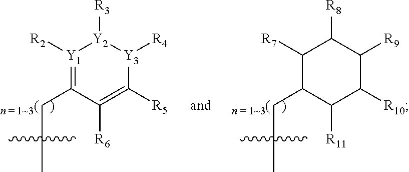
R₂ to Ru are independently selected from the group consisting of hydrogen, halogen, cycloalkyl, alkenyl, alkoxy, a substituted alkyl, and a substituted alkoxy;
X₁, X₂, Y₂, and Y₃ are independently selected from the group consisting of C and N, and Y₁ is C; and
R₁ is selected from the group consisting of phenyl, a substituted phenyl, pyrimidinyl, and pyridinyl.

2. The compound of claim 1, wherein R₂ to R₁₁ are independently a substituted C1-C8 alkyl.

3. The compound of claim 1, wherein R₂ to R₁₁ are independently selected from the group consisting of trifluoromethyl and trifluoromethoxy.

4. The compound of claim 1, wherein R₁ is selected from the group consisting of phenyl substituted with 0 to 5 R₁₂, the pyrimidinyl, and the pyridinyl; and R₁₂ is selected from the group consisting of the halogen, cyano, C1-C6 alkyl, C1-C6 alkenyl, C3-C9 cycloalkyl, C1-C6 haloalkyl, —NH₂, —NO₂, —SH, —OH, —CH₃COOH, —CH₂OH, a substituted C1-C6 alkoxy, C2-C6 alkynyl, an aldehyde group, and an ester group.

5. The compound of claim 4, wherein R₁₂ is —CF₃.

6. The compound of claim 4, wherein the compound has a structure shown in Formula I-1: 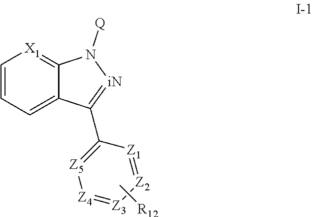
wherein
in the Formula I-1, Q is a structure being one selected from the group consisting of: 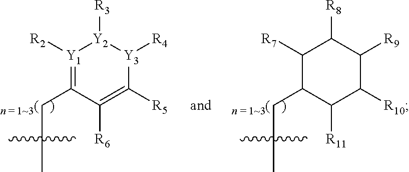
R₂ to R₁₁ are independently selected from the group consisting of the hydrogen, the halogen, the cycloalkyl, the alkenyl, the alkoxy, the substituted alkyl, and the substituted alkoxy;
R₁₂ is selected from the group consisting of the halogen, the cyano, the C1-C6 alkyl, the C1-C6 alkenyl, the C3-C9 cycloalkyl, the C1-C6 haloalkyl, —NH₂, —NO₂, —SH, —OH, —CH₃COOH, —CH₂OH, the substituted C1-C6 alkoxy, the C2-C6 alkynyl, the aldehyde group, and the ester group; and
X₁, X₂, Y₂, Y₃, Z₁, Z₂, Z₃, Z₄, and Z₅ are independently selected from the group consisting of C and N, and Y₁ is C.

7. The compound of claim 6, wherein R₂ to R₁₁ are independently selected from the group consisting of a substituted C1-C8 alkyl.

8. The compound of claim 6, wherein R₂ to R₁₁ are independently selected from the group consisting of trifluoromethyl and trifluoromethoxy.

9. The compound of claim 6, wherein R₁₂ is —CF₃.

10. The compound of claim 6, wherein Z₁ and Z₅ each are N, and Z₂ to Z₄ each are C; and the compound has a structure shown in Formula I-2:

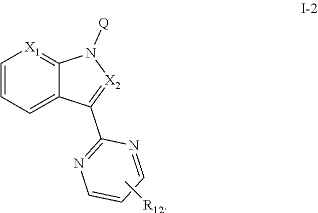

11. The compound of claim 10, wherein R₁₂ is selected from the group consisting of F, Cl, Br, methyl, ethyl, n-propyl, n-butyl, isopropyl, cyclopropyl, sec-butyl, cyclobutyl, cyclopentyl, isobutyl, 2-methylpentyl, allyl, 2-methylallyl, the cyano, —NH₂, —NO₂, —SH, —OH, —CH₃COOH, —CH₂OH, and —CH₃COOCH₃.

12. The compound of claim 10, wherein R₁₂ is one selected from the group consisting of:

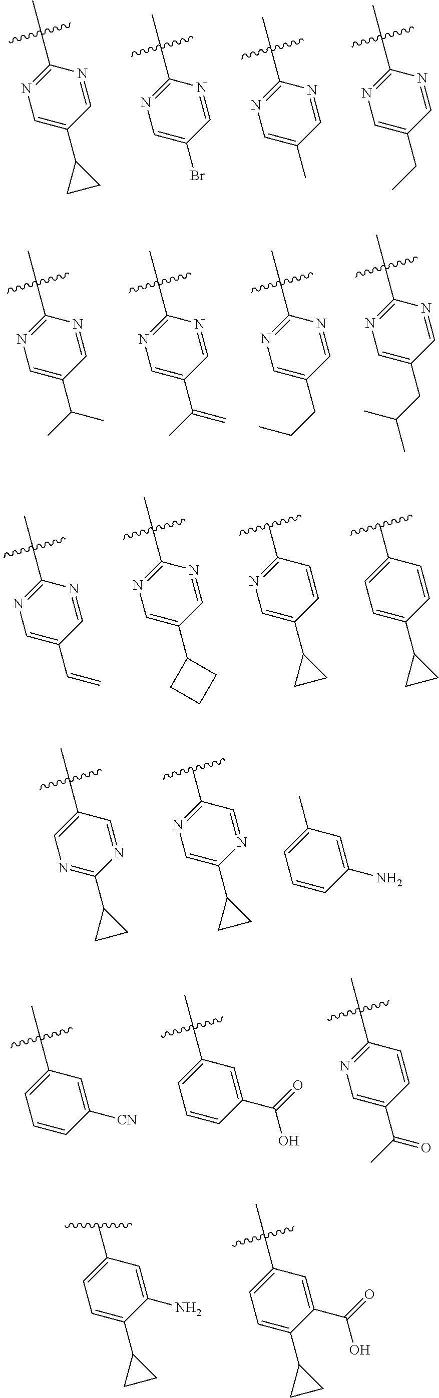

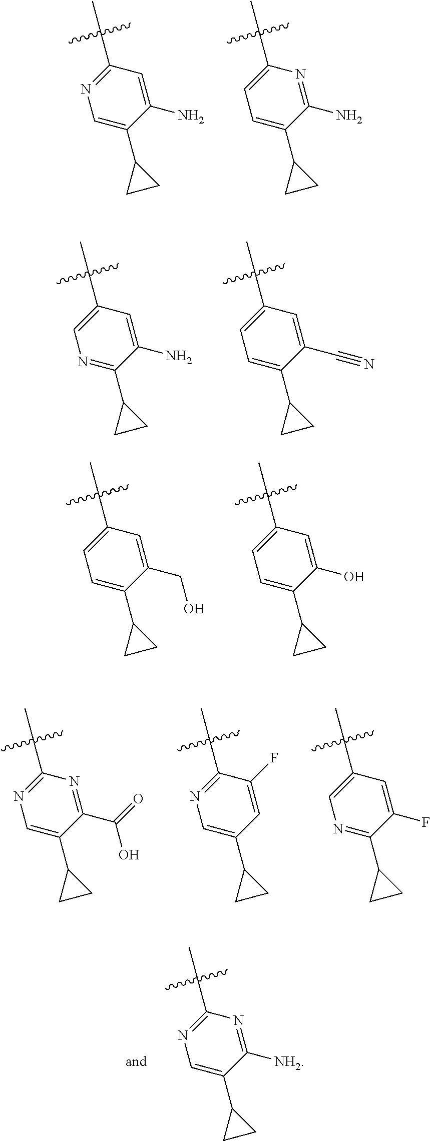

13. The compound of claim 10, wherein Q is one selected from the group consisting of:

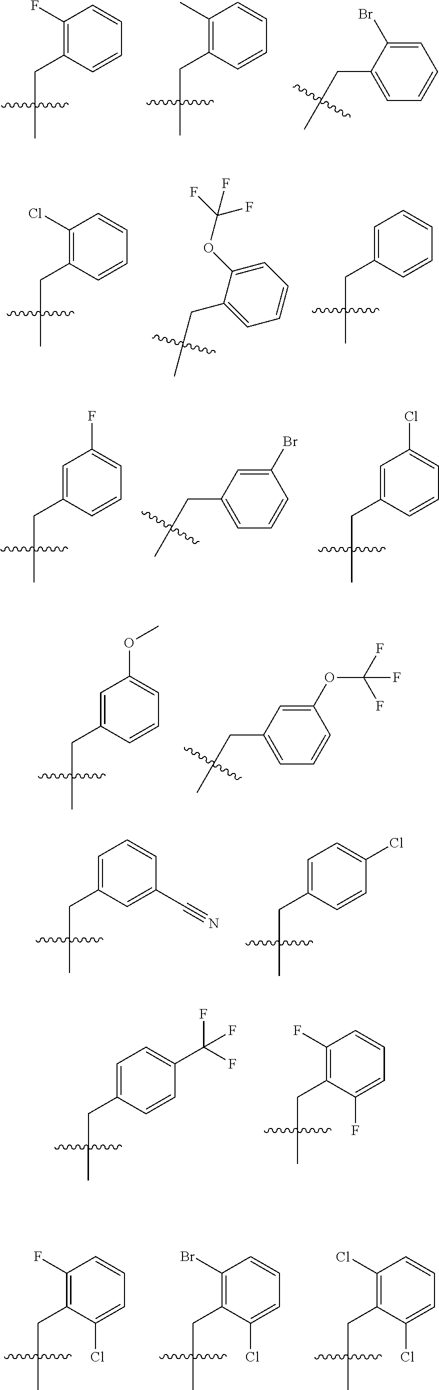

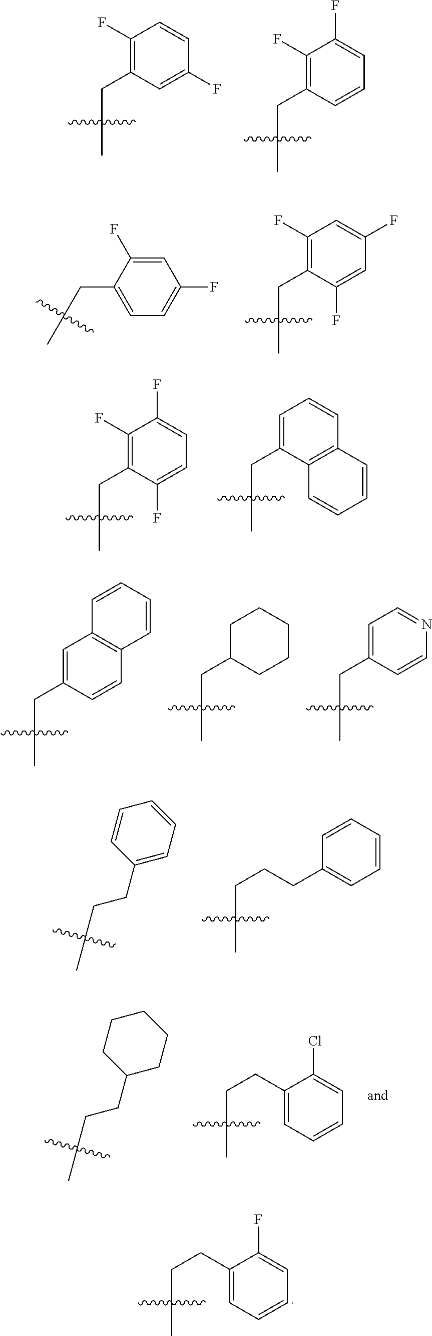

14. The compound of claim 1, wherein the compound has a structure being one selected from the group consisting of:

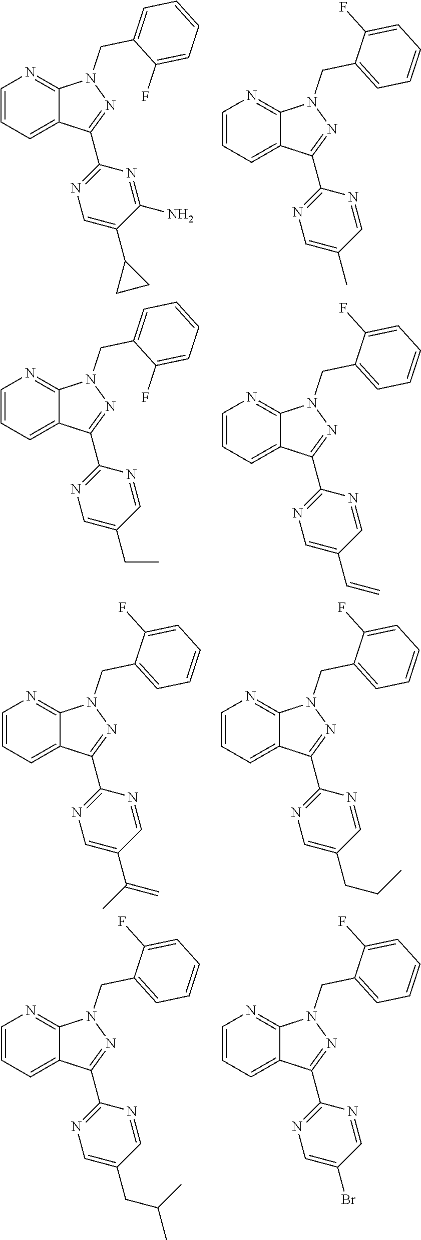

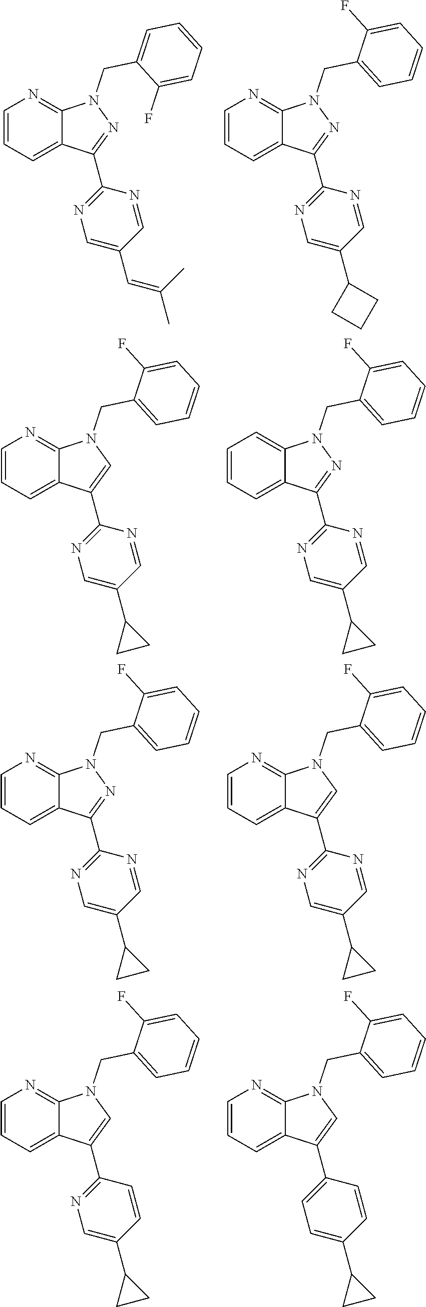

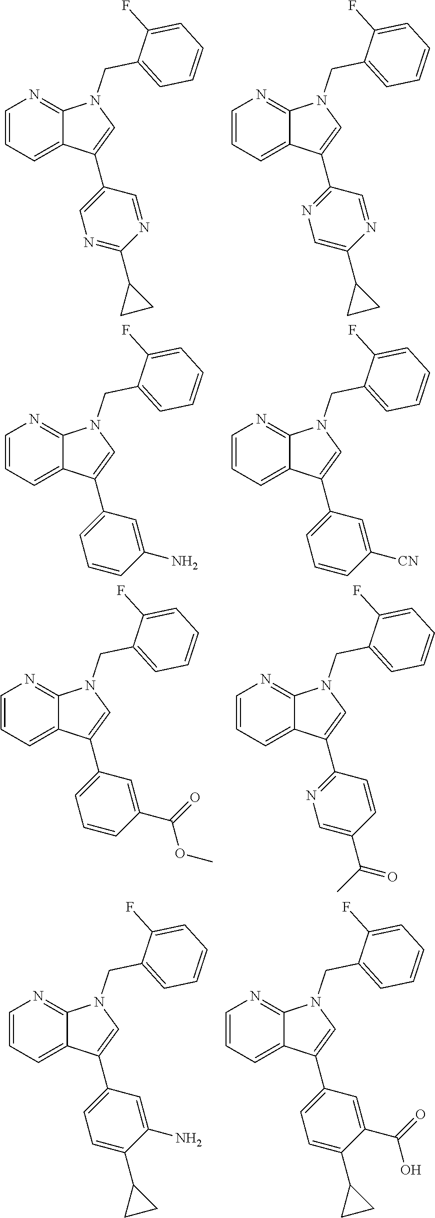

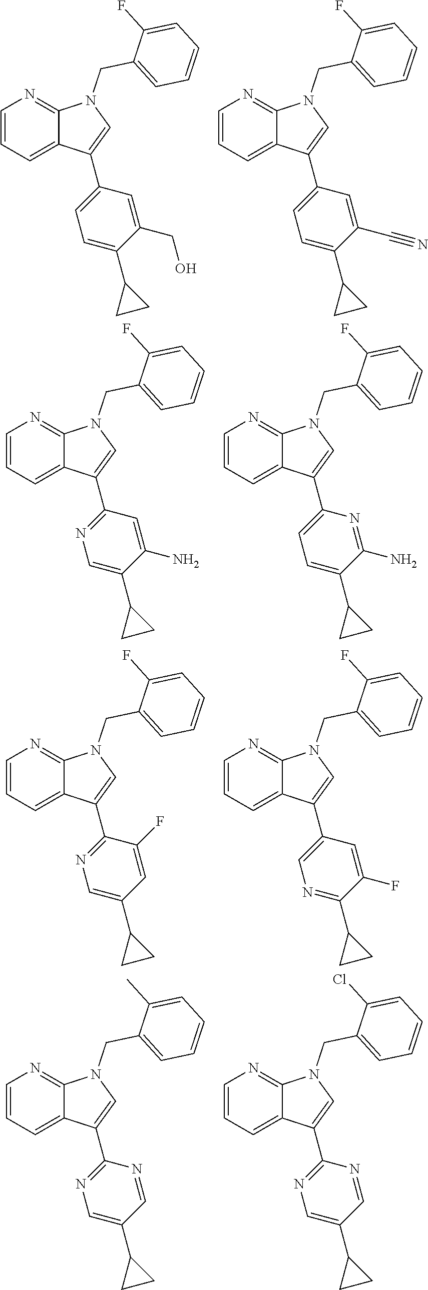

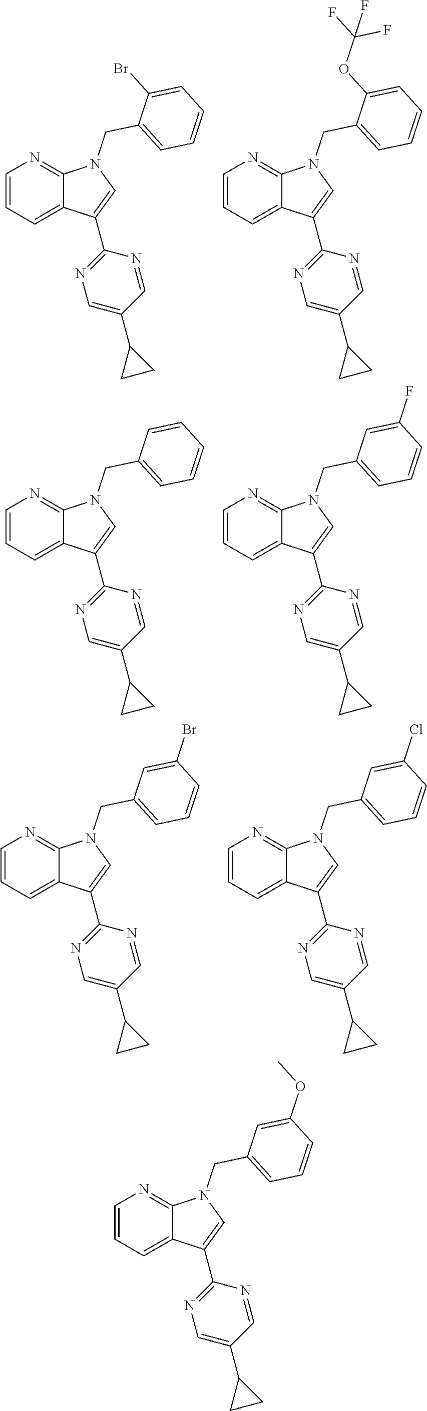

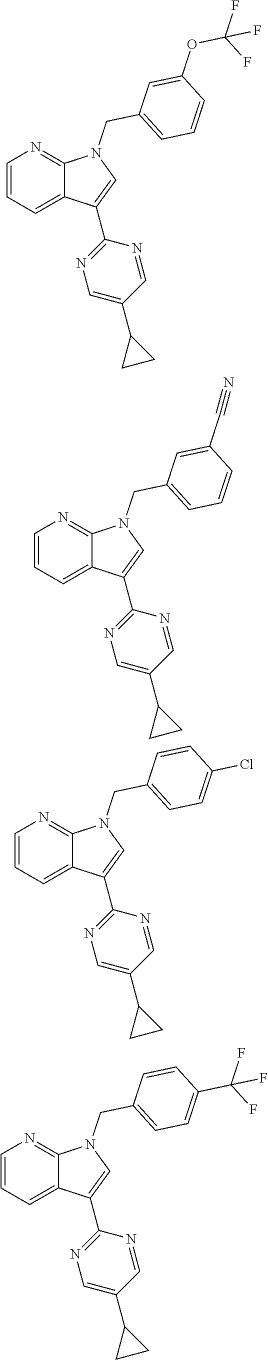

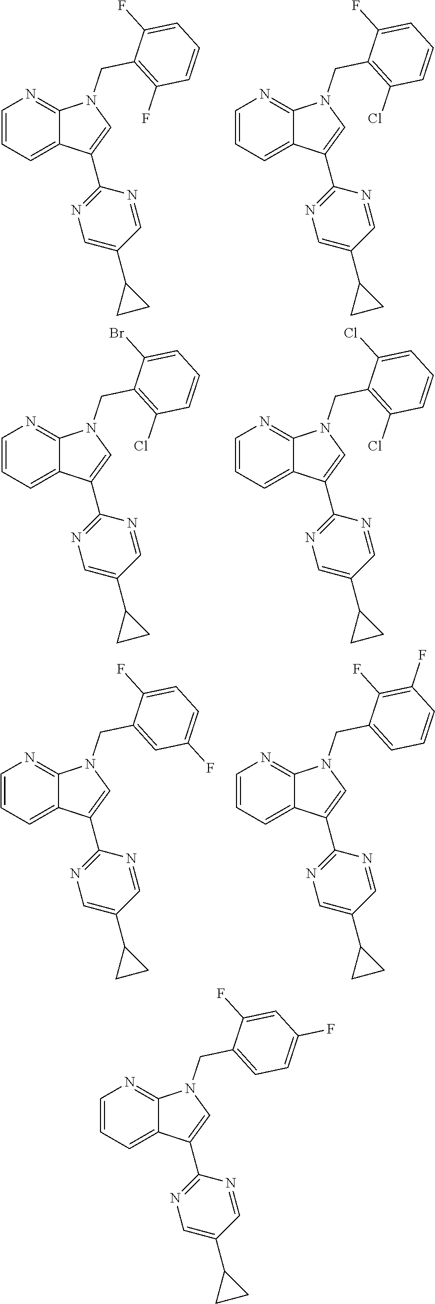

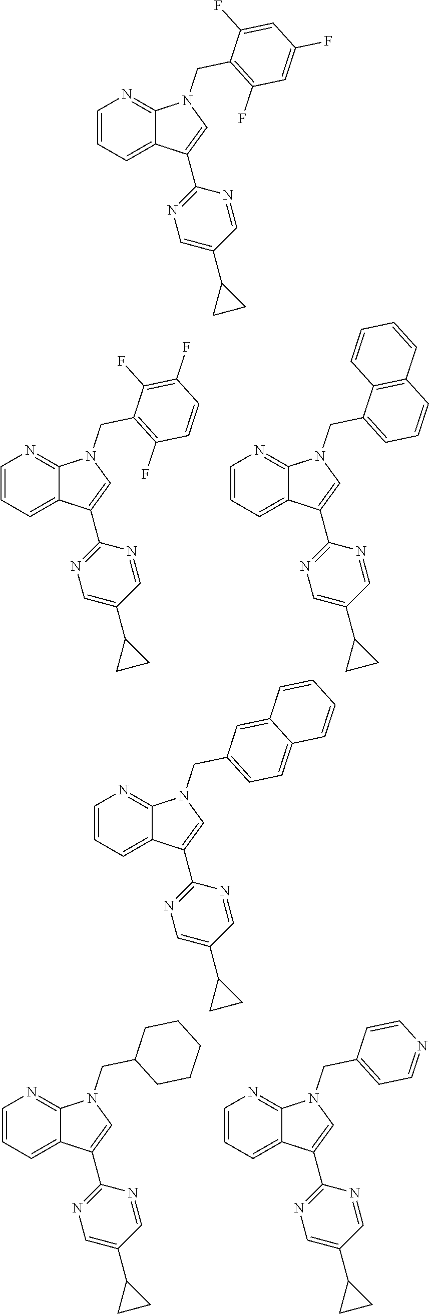

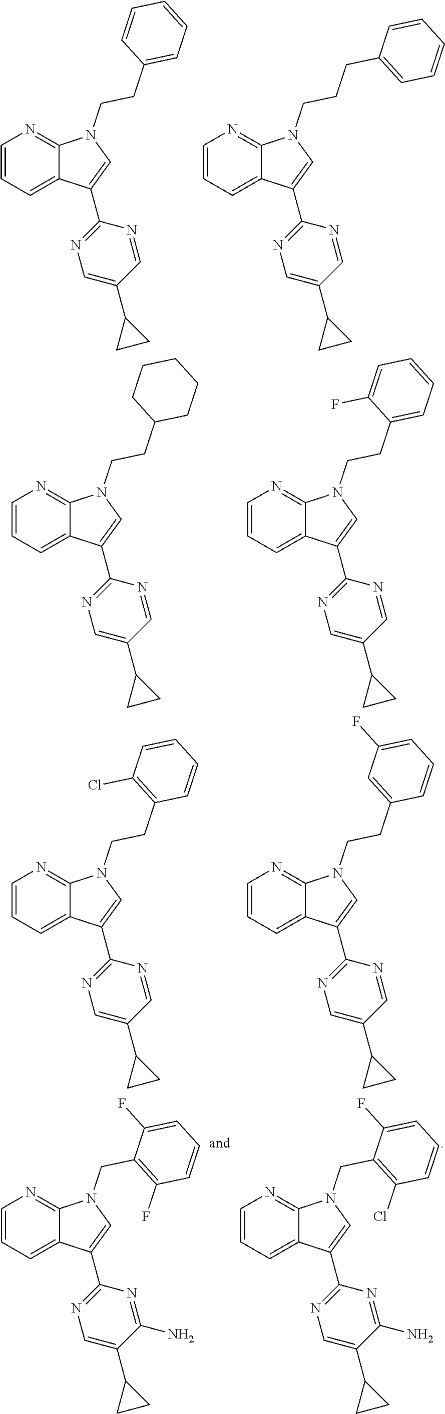

15. An isotopic substitute for one or more atoms, a pharmaceutically acceptable hydrate, a solvate or a polycrystal, and a salt of the compound of claim 1.

16. The isotopic substitute of claim 15, wherein an isotope in the isotopic substitute is deuterium.

17. (canceled)

18. (canceled)

19. A method for preparing the compound of claim 1, comprising the following steps:
(a) subjecting a starting material A and a halogen-substituted benzyl to reaction under an alkaline environment to obtain an intermediate B;
(b) subjecting the intermediate B and bis(pinacolato)diboron to first Suzuki coupling to obtain an intermediate C; and
(c) subjecting the intermediate C and a halogen-containing substituted phenyl, a substituted pyridinyl, or a substituted pyrimidinyl to second Suzuki coupling to obtain a final product D;
wherein the starting material A, the intermediate B, the intermediate C, and the final product D have the following structures, respectively: [chemical structure]
